(19) United States
(12) Patent Application Publication
(10) Pub. No.: US 2005/0288792 A1
(43) Pub. Date: Dec. 29, 2005

(54) Modular ankle prosthesis and associated method

(76) Inventors: Mark D. Landes, Warsaw, IN (US); D. Steven Block, Warsaw, IN (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/874,612

(22) Filed: Jun. 23, 2004

(51) Int. Cl. A61F 2/42

(52) U.S. Cl. 623/21.18

(57) ABSTRACT

An implant for use in ankle arthroplasty is provided. The implant includes a first member for cooperation with the tibia and a second member for cooperation with the talus. The second member is operably associated with the first member. The implant also includes a third member rigidly removably connectable to the second member. The third member includes a portion of the third member for attachment to the calcaneous. The third member is adapted to provide for a first position in the calcaneous when said third member is in a first relative position with respect to the second member is adapted to provide for a second position in the calcaneous when the third member is in a second relative position with respect to the second member.

### TECHNICAL FIELD OF THE INVENTION

The present invention relates generally to the field of orthopaedics, and more particularly, to an instrument for use in arthroplasty.

### BACKGROUND OF THE INVENTION

Prosthetic devices which are implanted for replacement of joints are well known. Such implants take the place of the body's own joints which fail, such as may be required for patients suffering from rheumatism, degenerative or traumatic arthritis, including osteoarthritis. A number of problems are associated with joint replacement. The joint should function in a manner, which simulates the natural joint, providing substantially the same degree of motion.

The ankle joint, or joint between the leg bones, tibia and fibula, and the talus, are frequently a source of osteo or rheumatoid arthritis. Typically, sufferers of rheumatoid and osteoarthritis at the ankle joint have been generally limited to a procedure called fusing. In a fusing procedure, the tibia, and typically the fibula, are fused or secured together with the talus to reduce the patient's pain and improve mobility. Clearly, the use of fusing does not provide the same degree of motion as a natural ankle joint.

For example, for ankle replacements, the joint should supply at least the same degree of motion as is required for walking. In addition, the joint should not occupy more space in the body than the natural joint. Problems arise in connection with the replacement joint to bone and tissue. The joint should also be as easy to implant as possible so that intricate operations are not required, thus reducing the chance of complications. The joints must have sufficient strength and durability to withstand the weight and stresses which are applied.

Ankle joints pose additional problems due to the weight supported and range of motion required for walking. Attachment of the tibia, which extends substantially vertically is difficult, as portions of the fibula may also be removed for implants. Matching the pivot point of the joint is critical, as misalignment can lead to difficulty in walking and other motions, which may cause the patient considerable pain.

The durability of a replacement joint is also important, as the ankle experiences high stresses during walking, running, and jumping, as well as fatigue over time. These stresses may crack or fracture ankle components of replacement joints, which absorb a substantial amount of the pressures during the aforementioned activities.

A particularly successful ankle implant for use in total ankle arthroscopy is disclosed in U.S. Pat. No. 5,326,365 to Alvine, and assigned to the same assignee as the instant application. U.S. Pat. No. 5,326,365 is hereby incorporated in its entirety by reference.

The total ankle implant, as disclosed in U.S. Pat. No. 5,326,365, is marketed by DePuy Orthopaedics, Inc. under the name Agility™ Ankle.

Currently designed ankle arthoplasty prosthesis perform quite well on patients having a primary or initial total ankle arthoplasty. Occasionally, however, the talus of the patient may be in such a condition that the talus does not provide sufficient support for the total ankle prosthesis.

An even more common problem with currently available total arthoplasty prostheses is the use of such prosthesis in a revision total ankle arthoplasty.

Referring now to FIG. 2, a prior art total ankle arthoplasty prosthesis 2 is shown in position on a patient's ankle 3. The ankle 3 may be, for example, an Agility ankle such that is made by DePuy Orthopaedics, Inc., Warsaw, Ind. As shown in FIG. 2, the ankle prosthesis 2 includes a talar component 4 which rests on the talus 5. The talus 5 may be machined to provide an accurate position for the prosthesis 2. Depending on physiological conditions of the patient and the progression of the osteoarthritis and the rheumatoid arthritis, as well as, the aging of the patient, the bone around the prosthesis 23 may deteriorate.

Referring now to FIG. 3, the prosthesis 2 is shown in position of the ankle 3 with the prosthesis 2 having subsided or moved downwardly or progressed further into the talus 5. The talar component 4 can thus move from its initial position 6A shown in phantom to its subsided position 7A as shown in solid.

Referring now to FIG. 4, the progression of osteoarthritis or rheumatoid arthritis or aging may eventually cause the prosthesis 2 to subside even further into the talus and may eventually pass through the talus 5 into calcaneus 8. As shown in FIG. 4, the talar component 4 of the prosthesis 2 may move from first position 6B as shown in phantom to the subsided position 7B as shown in solid. In its fully subsided position 7B the talar component 4 rests on the calcaneus 8.

It should be appreciated that with the subsidence of the prosthesis 2, the prosthesis 2 may become loosened with respect to the talus 2. The loosened components and the subsidence of the prosthesis 2 may result in bone loss and cause severe pain to the patient.

Subsidence of the prosthesis 2 may result in reduced motion of the ankle 3. For example and is shown in FIG. 5, the dorsiflexion of the ankle 3 may be reduced or limited from normal dorsiflexion as shown in position 9 in solid to a more limited dorsiflexion as shown in phantom as position 10.

Referring now to FIG. 6, the loosening and the subsidence of prosthesis 2 may result in loss of plantar flexion. For example and is shown in FIG. 6, the plantar flexion may be reduced from normal flexion as shown as position 11 to a much more limited plantar flexion as shown as position 12.

Further subsidence and loosening of the prosthesis 2 may limit the inversion and eversion movements of the ankle 3. For example, and as shown in FIG. 3 the eversion may be limited from normal eversion as shown as position 13 in phantom to a more limited eversion as shown as position 14 in solid. Similarly, the inversion may be limited from a normal inversion as shown as position 15 to a more limited inversion as is shown in solid as position 14.

The loosening and subsidence of the prosthesis 3 usually occurs with massive bone loss to the talus and as stated earlier, the prosthesis 2 may subside down into the calcaneus. The mere replacement of the original prosthesis with another larger component is generally is not successful in correcting the problem.

Attempts to address the revision of the total ankle arthoplasty have met with limited success. Typically once the primary ankle prosthesis has loosened and subsided the typical surgical procedure is to fuse the ankle. In such a procedure a metal rod is inserted through the calcaneus through the talus into the tibia to fuse or lock the talus to the tibia.

With some very limited success, some failed primary total ankle arthoplasty prosthesis have been replaced with a revision total ankle arthoplasty. The prosthesis for such procedures may need to be specially designed and specially built. These prostheses can be very specific expensive and provide the surgeon with only one very specific implant option in time of the surgery.

The implantation of such custom devices is often a very technically demanding procedure as instrumentation and surgical procedures are not well established. The present invention is directed to overcome at least some of the afore mentioned problems.

### SUMMARY OF THE INVENTION

According to the present invention, a total ankle arthoplasty prosthesis has been invented for use in revision of total ankle arthoplasty. The invention includes the use of talar components that are more effective for use of revision procedures. The present invention includes the use of stems that are designed for revision cases where the primary talar device has loosened or subsided.

The present invention may include a main talar component having an articular surface along with modular stems of various lengths and diameters. The stems may be fluted or may be porous coated. The prosthesis of the present invention may further include modular wedges or blocks which may be designed to be affixed to the inner portion of the main talar component to accommodate particular wear of the talus against the primary prosthesis.

The present invention may provide for a modular revision system for a primary ankle arthoplasty. The present invention may allow for a difficult revision of a talar component while obviating the need for a custom implant. The present invention may give the surgeon flexibility for ankle revision procedures.

Total ankle arthoplasty of the present invention may include various angular articulate blocks having different sizes and thickness. Also included may be various stems having different diameters and lengths. The stems may attached to the talar blocks during surgery in the operating room.

Trial prostheses including trial stems and blocks may be used to determine the component size and stem eversion angle based on the patient's unique anatomy. The talar block may be assembled to any modular stem to make either a right or a left hand assembly. The invention may also allow the surgeon to use modular blocks or wedges to fill voids left by bone deficiencies in the talus during subsidence.

According to one embodiment of the present invention, there is provided an implant for use in ankle arthroplasty. The implant includes a first member for cooperation with the tibia and a second member for cooperation with the talus. The second member is operably associated with the first member. The implant also includes a third member rigidly removably connectable to the second member. The third member includes a portion of the third member for attachment to the calcaneous. The third member is adapted to provide for a first position in the calcaneous when said third member is in a first relative position with respect to the second member is adapted to provide for a second position in the calcaneous when the third member is in a second relative position with respect to the second member.

According to yet another embodiment of the present invention there is provided a kit for use in assembling an implant for use in ankle arthroplasty. The kit includes a first member for cooperation with the tibia and a second member operably associated with the first member. The kit also includes a third member rigidly removably connectable to the second member. The third member includes a portion of the third member for attachment to the talus and a fourth member rigidly removably connectable to the second member. The fourth member includes a portion of the fourth member for attachment to the talus. The fourth member has at least one dimension different than that dimension of the third member.

According to still another embodiment of the present invention there is provided a talar component for use in an implant for use in ankle arthroplasty. The kit includes a first member for cooperation with the talus and a second member. The second member is rigidly removably connectable to the first member. The second member includes a portion of the second member for attachment to the calcaneous. The second member provides for a first position in the calcaneous when the second member is in a first relative position with respect to the first member and provides for a second position in the calcaneous when the second member is in a second relative position with respect to the first member.

According to a further embodiment of the present invention, there is provided a method for providing ankle arthroplasty. The method includes the steps of providing an ankle prosthesis kit including an tibial component, a bearing component, a talar articulating component, a first talar mounting component, and a second talar mounting component having at least one dimension different than the first talar mounting component, cutting an incision in the patient; preparing the talar cavity and the tibia cavity; implanting the tibial component into the tibial cavity; selecting one of the first talar mounting component and the second talar mounting component, implanting the selected one of the first talar mounting component and the second talar mounting component into the talar cavity, and positioning the bearing component between the tibial component and the selected one of the first talar mounting component and the second talar mounting component.

According to a yet another embodiment of the present invention, there is provided a trial for use with an implant having a first component, a second component and a third component for use in ankle arthroplasty. The trial includes a first trial member for cooperation with the tibia. The first trial member corresponds the first component. The trial also includes a second trial member operably associated with the first trial member. The second trial member corresponds to the second component. The trial further includes a third trial member rigidly removably connectable to the second member. The third trial member includes a portion thereof for attachment to the talus. The third trial member corresponds to the third component.

The technical advantages of the present invention include the ability of the present invention to provide a total ankle prosthesis for patients where the damaged to the talus prohibits such surgeries using prior art total ankle prostheses. For example, according to one aspect of the present invention a talar component for use in an implant for use in ankle arthoplasty is provided. Talar component includes a first member for cooperation with the tibia component and second member connectable to the first member. The second member includes a portion that may attach into the calcaneus. The calcaneus is not damaged by the subsidence into the prosthesis of the talus. The calcaneus can provide for a proper support for the revision total ankle arthoplasty prosthesis. Thus, the present invention provides for a total ankle arthoplasty prosthesis where a total ankle prosthesis may be prohibited due to damage to the talus.

The technical advantages of the present invention further include the ability of the total ankle arthoplasty prosthesis of the present invention to fill voids left by bone deficiencies in the talus. For example, according to one aspect of the present invention, an implant for use in the ankle arthoplasty is provided which includes a first member for cooperation with the tibia and a second or talar member for cooperation with the talus. The talar member includes augments operably associated with the second member. The augments may be in the form of modular proximal wedges, which may be utilized to fill voids left by the bone deficiencies in the talus. Thus, the present invention provides for the ability to fill voids that are left by bone deficiencies in the talus.

The technical advantages of the current invention further include the ability to use the implant of the present invention with different degrees of bone damage. For example, according to one aspect of the present invention, a kit for use in assembling an implant for use in ankle arthoplasty is provided. The kit includes a first talar member including a portion for attachment to the talus and a second talar member including a portion for attachment to the talus. The second talar member has at least one dimension longer than the dimension of the first talar member. Thus, the present invention provides for use on ankles with different degrees of bone damage.

The technical advantages of the present invention, further include the ability of the implant of the present invention to permit revisions to total ankle prostheses. According to another aspect of the present invention, an implant for use in ankle arthoplasty is provided including a first member in cooperation with the tibia and a second member. A bearing member is positioned between the first member and the second member. The second member includes a portion for cooperation with the calcaneus. Since the calcaneus will provide for sufficient bone support for a revision total ankle prosthesis, the present invention thus provides for the ability to provide for revision total ankle prostheses.

The technical advantages of the present invention further include the elimination of the need for right and left hand or side specific implants. For example, according to another aspect of the present invention, a total ankle prosthesis is provided having removably connectable components. The first component may be selective, rigid positioned with respect to the other component. Optionally indicia may be positioned on one or both of the first and second components so that the relative position may be easily adjusted or selected. Thus, the present invention for the elimination of the need for side specific implants.

The technical advantages of the present invention further include the ability to eliminate the need for a custom implant. For example, according to another aspect of the present invention a kit for use in an ankle athroplasty is provided. The kit may include a plurality of first members for cooperation with the tibia and a plurality of second members for cooperation with the talus. Any of the plurality of first members for cooperation with the tibia may be used with any of the second-members for cooperation with the talus. Thus the present invention can provide for a multitude of options such that the individual needs and variations in the anatomy of patients can be accomplished with the standard set of kit components thereby eliminating the need of a custom implants. Thus the present invention provides for the elimination of the need for custom implants.

The technical advantages of the present invention further include the ability to provide improved dorsiflexion and plantar flexion as well as eversion and inversion. For example, according to one aspect of the present invention, an implant for the use of total ankle arthoplasty is provided. The implant includes a first member for cooperation with the tibia and a second member including a portion for an attachment to the calcaneus.

A bearing is positioned between the first member and the second member for movable association with the first member and the second member. By providing a implant for the use in total ankle arthoplasty that includes a portion for attachment to the calcaneus, an implant can be provided that attaches to bone that is not seriously damaged during the loosening and subsidence of a primary total ankle implant. Thus, the present invention provides for a total ankle arthoplasty with improved range of motion for the patient.

The technical advantages of present invention include the ability to replace ankle fusion with a total ankle arthoplasty. For example and according to an embodiment of the present invention, an implant used in ankle arthoplasty is provided including a first member cooperation with the tibia and a second member to the calcaneus. A bearing is positioned between the first member and the second member. The attachment to the calcaneus provides for a secure attachment of the talar component even when the talus is severely damaged during the subsidence of a primary ankle implant. Thus, the present invention provides for a total ankle arthoplasty as an alternative to ankle fusion for revision surgery.

The technical advantages of the present invention, further include the ability to match an implant to a person's anatomy. For example, according to an embodiment of the present invention a kit is provided for use in ankle arthoplasty. The kit includes a plurality of first members for cooperation with a tibia and a plurality of second members for cooperation with a talus. Each of the first members for the cooperation with tibia may be operably associated with any of the other second members for attachment to the talus. Thus a plurality of components may be utilized to accommodate variations in anatomy of patients. Thus the present invention provides for the ability to match an implant to a person's anatomy.

The technical advantages of the present invention also include intra-operably optimized revision talar assembly. For example, according to an aspect of a present invention the kit for use in the assembly of an implant for use in ankle arthoplasty may include a plurality of trials for association with the tibia and plurality of trials for association with the talus. Any of the plurality of components for association with the tibia may be assembled with any of the components for cooperation with the talus such that intra-operably an optimum revision talar assembly may be provided.

Other technical advantages of the present invention will be readily apparent to one skilled in the art from the following figures, descriptions and claims.

### BRIEF DESCRIPTION OF THE DRAWINGS

For a more complete understanding of the present invention and the advantages thereof, reference is now made to the following description taken in connection with the accompanying drawings, in which:

FIG. 1 is a plan view partially in cross section of a prosthesis implanted in a patient in accordance with an embodiment of the present invention;

FIG. 2 is a plan view partially in cross section of a prior art prosthesis implanted in a patient;

FIG. 3 is a plan view partially in cross section of the prior art prosthesis of FIG. 1 showing subsidence in the talus;

FIG. 4 is a plan view partially in cross section of the prior art prosthesis of FIG. 1 showing subsidence into the calcaneus;

FIG. 5 is a plan view of the foot of a patient in dorsiflexion;

FIG. 6 is a plan view of the foot of a patient in plantar flexion;

FIG. 7 is a top view of the foot of a patient in inversion in solid and in eversion in phantom;

FIG. 8 is an end view partially in cross section of the prosthesis of FIG. 1 implanted in a patient;

FIG. 9 is an enlarged plan view partially in cross section of the prosthesis of FIG. 1;

FIG. 10 is an enlarged end view partially in cross section of the prosthesis of FIG. 1;

FIG. 11 is a plan view partially in cross section of the prosthesis of FIG. 1 showing the talar component in three positions relative to the tibial component;

FIG. 12 is a perspective view of the talar component of the prosthesis of FIG. 1;

FIG. 13 is a plan view of the talar component of the prosthesis of FIG. 1;

FIG. 13A is an end view of the talar component of the prosthesis of FIG. 1;

FIG. 14 is an enlarged plan view of the stem of the talar component of the prosthesis of FIG. 1;

FIG. 14A is an enlarged plan view of another embodiment of the present invention showing a one piece talar component;

FIG. 15 is an exploded plan view of in accordance with another embodiment of the present invention the embodiment of the present invention in the form of a prosthesis including an augment;

FIG. 15A is a plan view of a prosthesis in accordance to another embodiment of the present invention in the form of a talar component with augments that fit over the talar component;

FIG. 15B is a plan view of a prosthesis in accordance to another embodiment of the present invention in the form of a talar component with augments that are attached to the talar component;

FIG. 16 is a plan view of in accordance with another embodiment of the present invention in the form of a prosthesis including a cylindrical connection on the stem;

FIG. 17 is a plan view of in accordance with another embodiment of the present invention in the form of a prosthesis including a threaded connection on the stem;

FIG. 18 is a plan view of in accordance with another embodiment of the present invention in the form of a prosthesis including indicia for orienting the stem;

FIG. 19 is a partial enlarged bottom view of the stem of the talar component of the prosthesis of FIG. 18;

FIG. 20 is an exploded plan view in accordance with another embodiment of the present invention in the form of a prosthesis including a reverse taper connection of the stem to the prosthesis;

FIG. 21 is a plan view partially in cross section of a trial prosthesis for use with the prosthesis of FIG. 1;

FIG. 22 is an exploded plan view of the talar component of the trial prosthesis of FIG. 21;

FIG. 23 is a plan view of a kit for use in performing total ankle arthroplasty in accordance with an embodiment of the present invention;

FIG. 24 is a flow chart of a method for performing total ankle arthroplasty in accordance with another embodiment of the present invention;

FIG. 25 is an exploded plan view of a prosthesis in accordance with yet another embodiment of the present invention in the form of a prosthesis including a spline and a fastener;

FIG. 26 is an partial top view of the prosthesis of FIG. 25 showing the spline in greater detail

FIG. 27 is an bottom view of the prosthesis of FIG. 25 showing the indicia in greater detail;

FIG. 28 is a plan view partially in cross section of a prosthesis trail for use with the prosthesis of FIG. 25;

FIG. 29 is a plan view of a prosthesis in accordance with a further embodiment of the present invention in the form of a prosthesis including a tapered connection, a support skirt and a fastener;

FIG. 30 is a plan view of a prosthesis in accordance with yet another embodiment of the present invention in the form of a prosthesis including a tapered connection, a support skirt, a dovetail lock and a fastener;

FIG. 31 is an exploded plan view of a prosthesis in accordance with a further embodiment of the present invention in the form of a prosthesis including a spline connection, a support skirt, a dovetail lock and a fastener;

FIG. 32 is a plan view of a prosthesis in accordance with yet another embodiment of the present invention in the form of a prosthesis including a cylindrical connection, a two piece talar component, a dovetail lock and a fastener;

FIG. 33 is a bottom view of the prosthesis of FIG. 32 showing the spline in greater detail; and

FIG. 34 is an end view of the prosthesis of FIG. 32 showing the dovetail lock in greater detail.

### DETAILED DESCRIPTION OF THE INVENTION

Embodiments of the present invention and the advantages thereof are best understood by referring to the following descriptions and drawings, wherein like numerals are used for like and corresponding parts of the drawings.

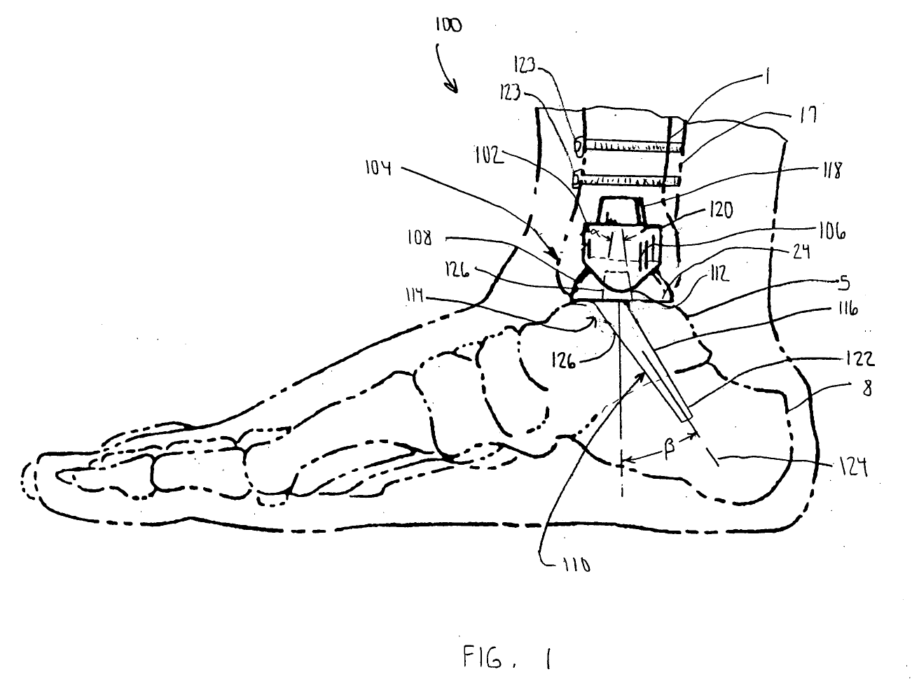

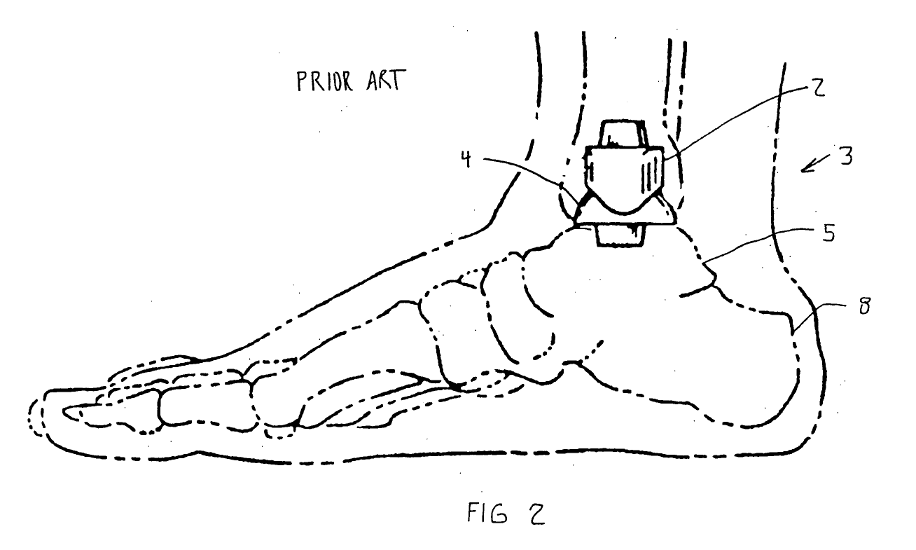

According to the present invention and referring now to FIG. 1, an implant 100 is shown in use for total ankle arthoplasty. The implant 100 includes a first member 102 for cooperation with the tibia 1. The first member 102 may also cooperate with fibula 17. It should be appreciated the tibia 1 and the fibula 17 may be secured to each other by, for example, cancellous or cortical screws 123.

Implant 100 may further include a talar assembly 104. Talar assembly 104 is adapted for cooperation with the talus 5. Talar assembly 104 may further cooperate with first member 102 to provide the freedom of motion required for the implant 100. Alternatively, a component may be positioned between the talar assembly 104 and the first member 102. For example, a bearing 106 may be operably positioned between the talar assembly 104 and the first member 102. The bearing 106 may be made of any suitable, durable material that is sterilizable and is compatible to the human body.

The bearing 106 may for example may be made of a plastic, a metal, or a composite. For example, the bearing 104 may be made of a metal, for example, cobalt chromium alloy, a titanium alloy, and a stainless steel alloy. The bearing 106 may be made of a plastic, for example an ultra-high molecular weight polyethylene. For example, the bearing 104 may be made of a crosslinked ultra-high molecular weight polyethylene such as Marathon® a product of DePuy Orthopaedics, Inc., Warsaw, Ind.

Shown in FIG. 1, the talar assembly 104 may include an articulating member 108 and an anchoring member 110. The articulating member 108 is operably associated with the first member 102. For example and is shown in FIG. 1, the articulating member 108 cooperates with the bearing 106, which cooperates with the first member 102. Therefore, the articulating member 108 is operably associated with the first member 102. It should be appreciated that the first member 102 and the articulating member 108 may directly contact each other or be operably associated to provide for the range of motion of the ankle 3.

Anchoring member 110 as shown in FIG. 1 is rigidly connectable to the articulating member 108. The articulating member 108 and the anchoring member 110 may be rigidly, movably, and connectable to each other in any suitable fashion. For example and as is shown in FIG. 1, the anchoring member 110 may include a protrusion 112 which fits within a cavity 114 formed in the articulating member 108. The anchoring member 110 includes a portion 116 of the anchoring member 110 for attachment to the talus 5.

It should be appreciated that while the anchoring member 110 may include the protrusion 112 and the articulating member 108 may include the cavity 114, alternative configurations for rigidly removable connection of the articulating member 108 to the anchoring member 110 may be provided. For example, the articulating member 108 may include a protrusion (not shown) which cooperates with a cavity (not shown) formed in the anchoring member 110.

The protrusion 112 of the anchoring member 110 may have any suitable shape and for example and as shown in FIG. 1 may be in the form of a male or external taper. For example and as shown in FIG. 1, the external taper of the protrusion 112 may be conofrustical. The protrusion 112 may form an included angle α for example, 0 to 20 degrees. Angle α may be selected to provide for a self-locking taper.

As shown in FIG. 1, the implant 100 may further include a tibia connecting member 118 operably associated with the first member 102. The tibia connecting member 118 may be in the form of a protrusion extending outwardly from the support face 120 of the first member 102. Tibia connecting member 118 may be generally planar with a slight taper for optimum support of the implant with minimal weakening of the tibia 1.

As shown in FIG. 1, the anchoring member 110 may include a portion 122 thereof for attachment to the calcaneus 8. The extension of the anchoring member 110 into the calcaneus 8 is helpful in obtaining rigid support for the articulating member 108. The portion 122 is particularly helpful when the primary implant, which has been removed from the patient to implant the revision implant 100, has subsided and or has created considerable bone loss to the talus 5.

The anchoring member 110 may have any suitable size and shape and may for example be general linear and may for example may have any suitable cross-section such as polygon, square, triangular, or round. The anchoring member 110 may be of uniform cross section or may be tapered.

The anchoring member 110 as is shown in FIG. 1, includes a first portion 124 extending from the articulating member 108 and having a longitudinal centerline 124 and the calcaneous portion 122 extending from the first section 126. The first section 126 extends posteriorly at an angle β from articulating member 108 in order that the first section 122 is positioned into the center position of the calcaneus 8.

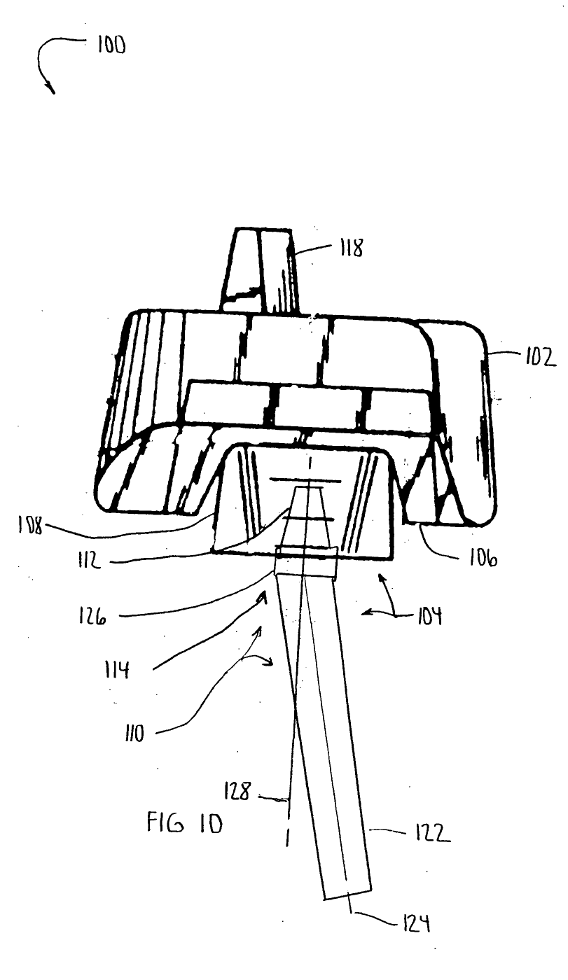

Referring now to FIG. 8, the anterior view of the implant 100 is shown. As shown in FIG. 8 the implant 100 is positioned on a right ankle. As shown in FIG. 8 the anchoring member 110 of the talar assembly 104 of the implant 100 is positioned such that calcaneous portion 122 of the anchoring member 110 extends from second section 126 of the anchoring member 110 at an angle θ, for example 0 to 20 degrees with respect to vertical centerline 119. It should be appreciated that the left ankle should be a mirror image of the right ankle in that the angle θ should be in the opposite direction.

Referring now to FIG. 9, the enlarged view of the implant 100 is shown. The implant 100 includes the first member 102 in cooperation with the tibia 1 as well as a talar assembly 104 with cooperation with the talus 5. The implant 100 may also include a bearing 106 positioned between the first member 102 and the talar assembly 104. Preferably the implant 100 is configured to provide for the general motions that the natural ankle can accommodate.

For example, the prosthesis 100 preferably allows for the dorsiflexion, planar flexion, eversion, and inversion. Preferably the implant 100 will also provide rocking or articulation medially and laterally. It should be appreciated that the articulating surfaces of the first member 102, the bearing 106, and the talar assembly 104 may be designed with any of a number of articulation shapes and configurations to accommodate the nature of the articulation available from a natural ankle.

As shown in FIG. 9, the first member 102, the bearing 106, and the articulating surfaces of the talar assembly 104 are similar to that of the Agility ankle sold by DePuy Orthopaedics, Inc., Warsaw, Ind. and generally described in U.S. Pat. No. 5,326,365. The first member 102 may include the tibia connecting member 118. The tibia connecting member 118 may have any suitable size and shape assisting in the anchoring of the first member 102 to the tibia 1.

The components of the implant 100 may be made of any suitable, durable material and may be made of, for example, a composite, a plastic, a ceramic, or a metal. Preferably the components of implant 100 are made of a suitable, durable material that is compatable with a commercially available sterilization techniques such as gas sterilization or gamma irradiation.

The first member 102, the talar assembly 104 including the anchoring member 110, and the articulating member 108 are preferably made of a durable metal. For example, the first member 102, the anchoring member 110, and the articulating member 108 may be made of, for example, a titanium alloy, a cobalt chromium alloy, or a stainless alloy.

The anchoring member 110 may include a first portion 126 and a calcaneous portion 122. The first portion 126 may have a first portion centerline 124 while the calcaneous portion 122 may have a second portion longitudinal centerline 128. It should be appreciated that the centerlines 124 and 128 may be coincident as shown in FIG. 9 and form included angle, for example, β with vertical centerline 119. The angle of β is preferably selected to engage the calcaneus 8. For example, the angle β may be for example 2 to 30 degrees.

It should be appreciated that the anchoring member 110 should be provided with the angle β as that the cavity 114 in the articulating member 108 is positioned with the protrusion 112 of the anchoring member 110 is in alignment with the calcaneus 8.

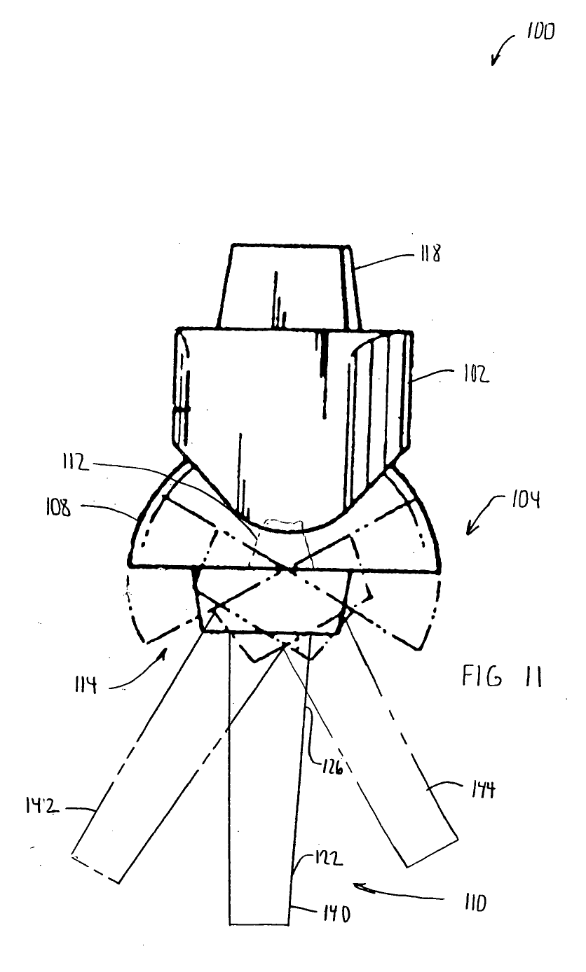

Referring now to FIG. 10 the implant 100 is shown in greater detail. The implant 100 includes the first member 102 and the bearing 106, and talar assembly 104.

Referring now to FIG. 11, the prosthesis or implant 100 is shown with the talar assembly 108 in various articulating positions. For example, and is shown in FIG. 11 the talar assembly 104 is shown in a first position 140 in solid. The prosthesis 100 is also shown in FIG. 11 in talar assembly 104 in a second position 142 as shown in dash lines. The prosthesis 100 is also shown in FIG. 11 with the talar 104 in third position 144 as shown in phantom. It should be appreciated that the prosthesis 100 may be capable of providing for the talar assembly 104 to be positioned in, for example, any position between the extremes of the second position 142 to the third position 144. It should also be appreciated that the prosthesis 100 may be designed to provide for a second position 142 and a third position 144 with even greater motion that is shown in FIG. 11.

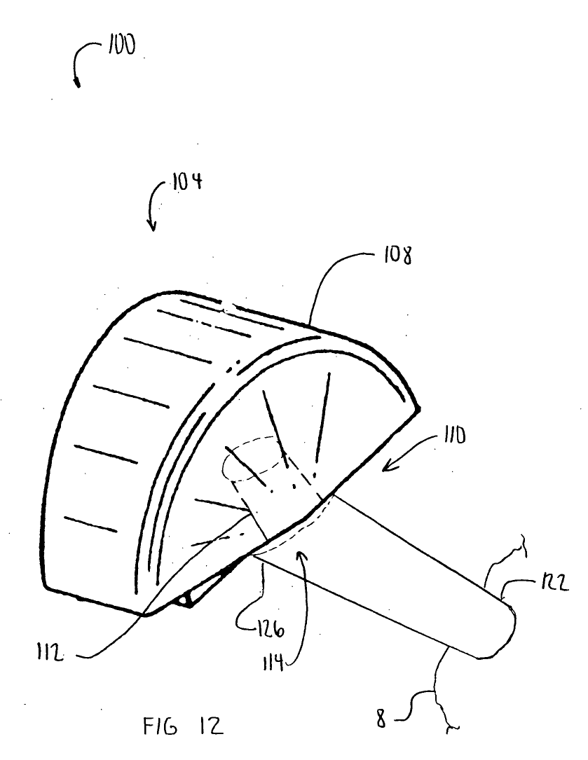

Referring now to FIG. 12, the prosthesis 100 is shown in a prospective view. The prosthesis 100 includes talar assembly 104 having both an articulating member 108 and an anchoring member 110. The first section 122 of the anchoring member 110 may include a portion for engagement with calcaneus 8.

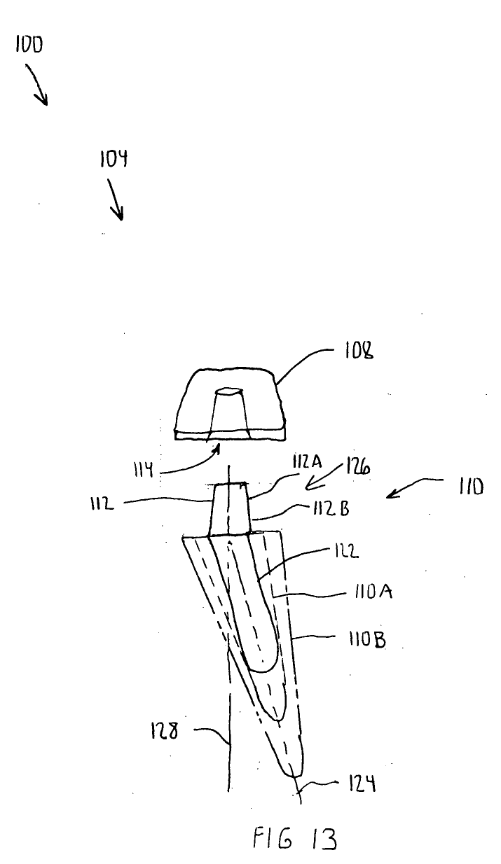

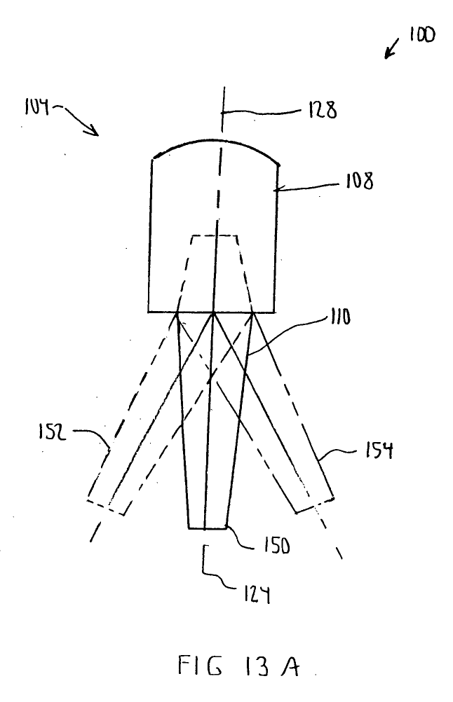

Referring now to FIG. 13, the prosthesis or implant is shown in an exploded position. The prosthesis 108 includes a talar assembly 104. The talar assembly 104 includes the anchoring member 108 as well as the anchoring member 110.

As shown in FIG. 13, the anchoring member 100 may have any suitable length capable of sufficient anchoring into the calcaneus 8. For example and is shown in FIG. 13, the anchoring member 110 of the talar assembly 104 of the prosthesis may have an additional length and diameter as shown as articulating member 110A as shown in the dashed lines.

Alternatively, the articulating member 110 may have an even larger length and a greater diameter and as shown in phantom as anchoring member 110B. It should be appreciated preferably each of the articulating members 110, 110A, and 110B have a protrusion that is substantially the same.

Therefore the protrusions 112, 112A, and 112B are identical in order that each of the protrusions 112, 112A, and 112B may mattingly fit into the cavity 114 in the articulating member 108. By designing a common protrusion each of the anchoring members 110, 110A, and 110B may be mattingly fitted with the articulating member 108 to provide for a variety of prosthesis options for the surgeon during the surgery.

According to an aspect of the present invention, the prosthesis may be utilized as a right ankle prosthesis or as a left ankle prosthesis for either the right foot or the left foot, respectively. For example and is shown in FIG. 13A, the prosthesis 100 may include the talar assembly 104 having the articulating member 108 and the anchoring member 110. It should be appreciated that in the medial lateral plane as shown in FIG. 13A, the centerline 128 of the articulating member 108 may be generally in line with centerline 124 of the anchoring member 110. It should be appreciated also that for a right or left hand prosthesis it may be desirable to have the anchoring member 110 extend medially or laterally from the articulating member 108 in order to position the anchoring member 110 centrally in the calcaneus. Therefore as shown in FIG. 13A for a left foot, the anchoring member 110 may be positioned centrally as shown in solid in position 150, medially as shown dashed lines in position 152 or laterally as shown in phantom as position 154.

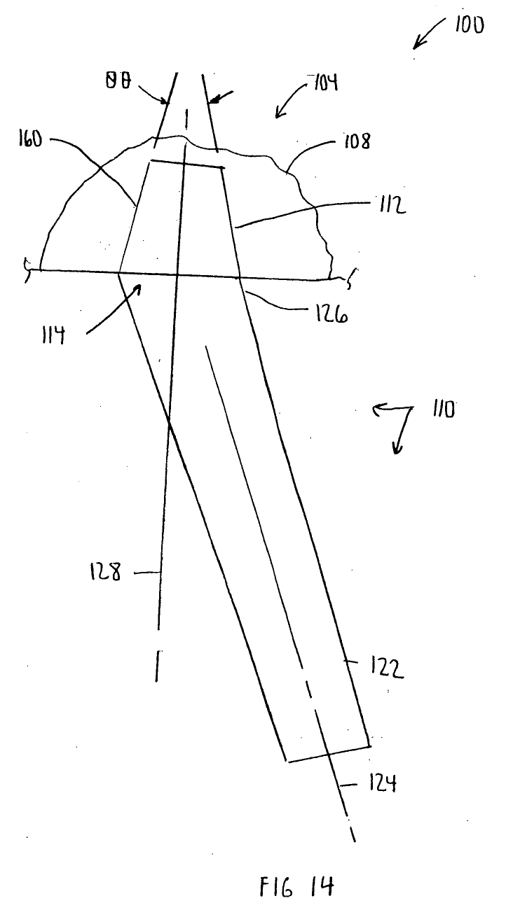

Referring now to FIG. 14, the anchoring member 110 is shown in greater detail. As shown in FIG. 14 the anchoring member 110 includes the first portion 122 for cooperation with the calcaneus and the second portion 126 including a protrusion 114 for cooperation with the cavity 128 formed in the articulating component 108. Protrusion 114 may include a tapered periphery 160 defining an included angle θθ for example 0 to 25 degrees. Preferably the angle θθ is selected to provide for a self-locking taper to secure the protrusion 112 to the cavity 114.

It should be appreciated that while a tapered protrusion may provide for a satisfactory connection between the articulating component 108 and the anchoring member 110, a multitude of alternate connections are possible in the spirit of the present invention.

It should be appreciated that a modular construction of the assembly will provide for right and left ankle prosthesis with the same components, alternate components. Constructions of the implant are possible. For example and is shown in FIG. 14A, another embodiment of the present invention is shown as prosthesis 200. Prosthesis 200 includes a first member or tibia member 202. The tibia member 202 is similar to the first member 102 of the prosthesis 100 of FIGS. 8-13. The prosthesis 200 further includes a talar component 204. A bearing 206 similar to the bearing 106 of the implant 100 in FIGS. 8-13 may be positioned between the tibia component and the talar component 204.

The talar component 104 may have the same general shape and configuration of the talar assembly 104 of the prosthesis 100 of FIGS. 8-13, but unlike the talar assembly 104 the talar component 204 is unitary or of a one piece construction. The talar component 204 includes an articulating portion 208 and anchoring portion 210, which extends into the calcaneus 8.

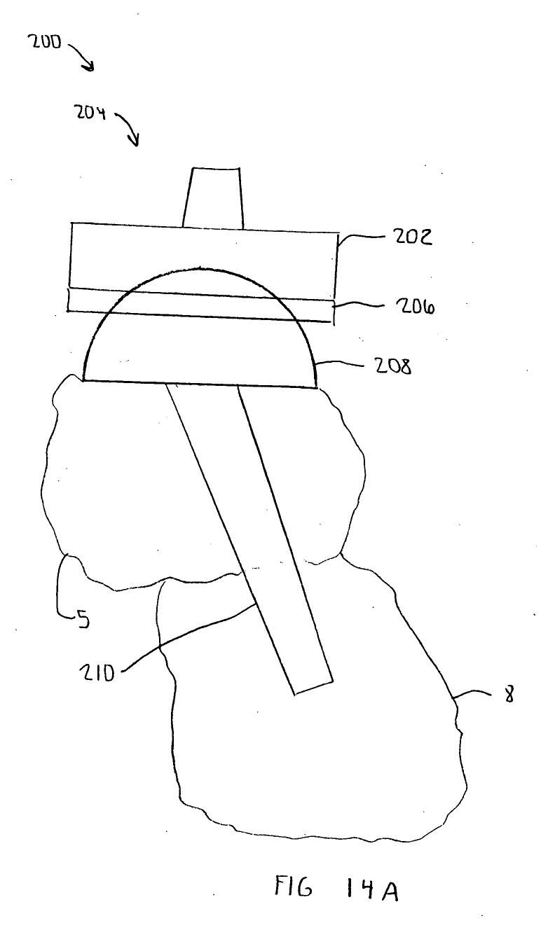

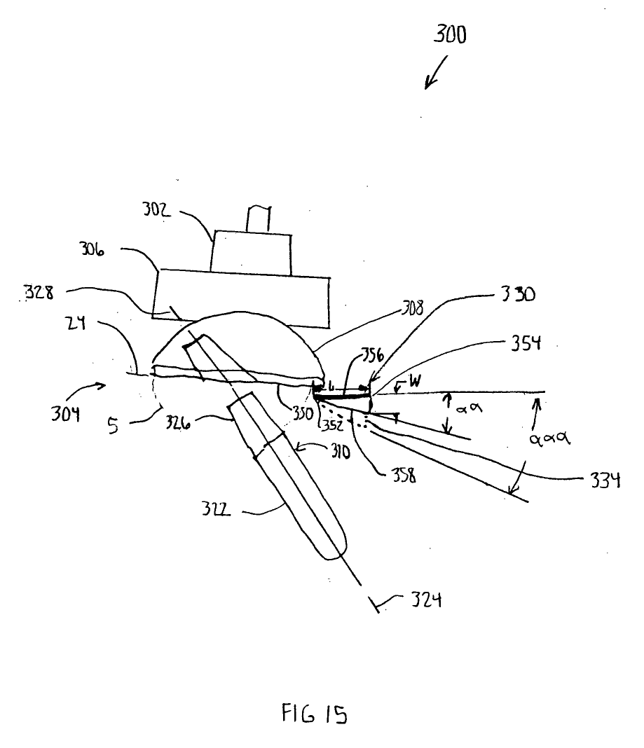

Referring now to FIG. 15, another embodiment of the present invention is shown as prosthesis 300. Prosthesis 300 is similar to the prosthesis 100 of FIGS. 8-13 and includes a tibia component 302 similar to the tibia component 102 of the prosthesis 100 of FIGS. 8-13. The prosthesis 300 further includes a talar assembly 304 somewhat similar to the talar assembly 104 of the prosthesis 100 of FIGS. 8-13.

The prosthesis 300 may include a bearing 306 positioned between the tibia component 302 and the talar assembly 304. The bearing 306 may be similar to the bearing 106 of the prosthesis 100 of FIGS. 8-13.

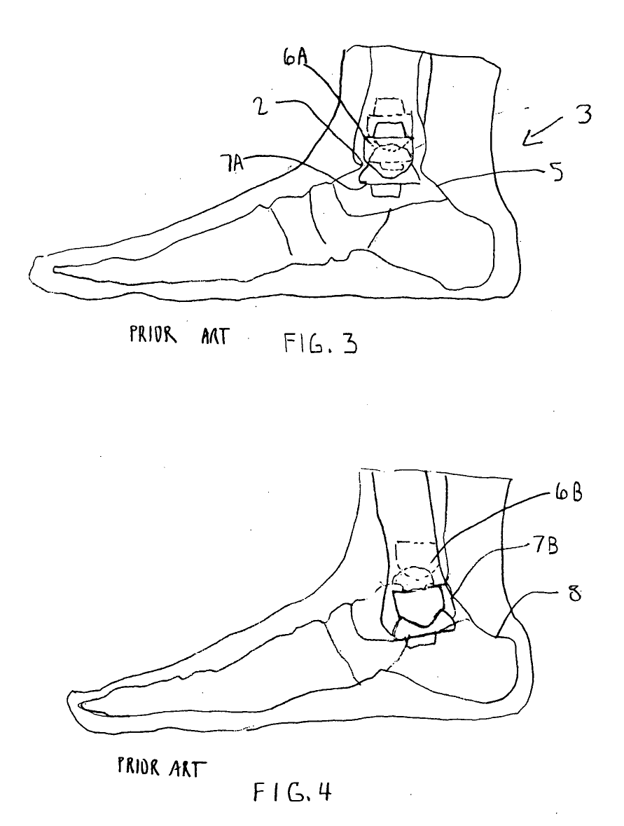

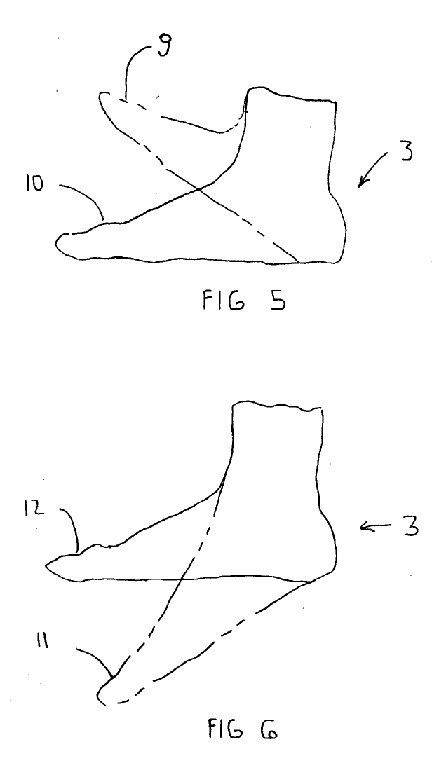

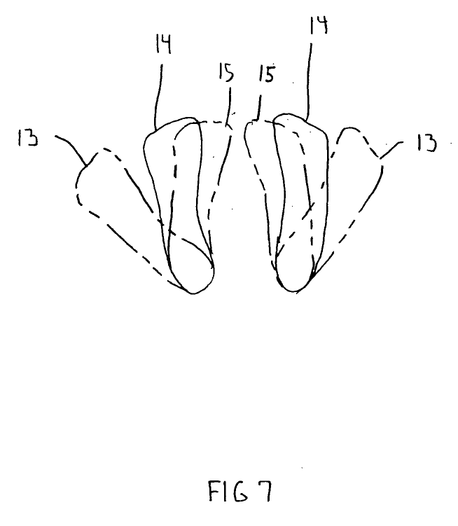

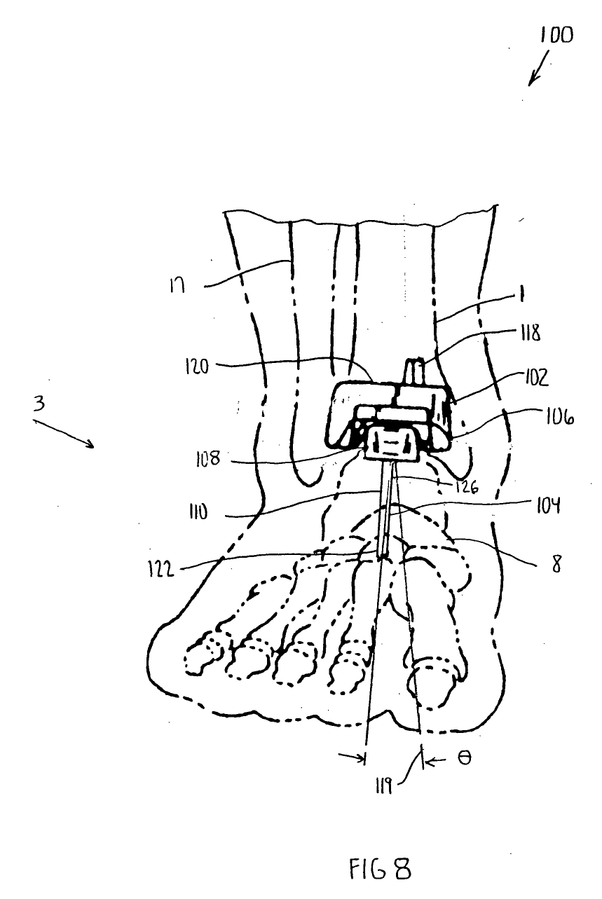

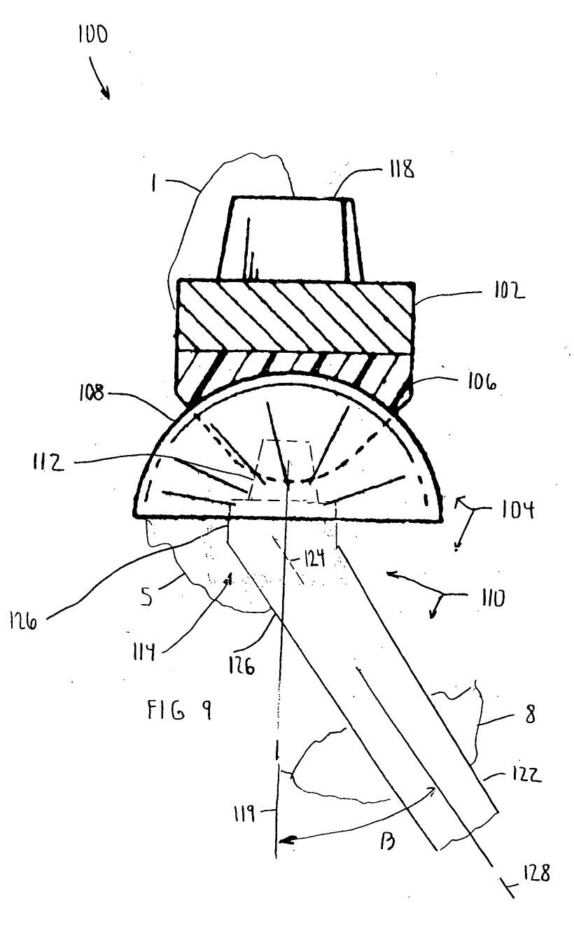

As shown in FIG. 15, the talar component 304 of the prosthesis 300 includes an articulating member 308 which rests upon the support surface 24 of the talus 5. The talar assembly 304 further includes an anchoring member 310 rigidly and removably connectable to the articulating member 308. The articulating member 308 is similar to the articulating 108 of the prosthesis 100 and the anchoring member 310 is similar to the anchoring member 100 of the prosthesis 100 of FIGS. 3-18.

The prosthesis 300 further includes a supporting feature 330 to provide support between support surface 350 of the articulating member 308 and the support surface 24 of the talus 5. The supporting feature 330 may have any suitable size and shape. For example, the supporting feature 330 may be in the form of an augment. The augment 330 may for example, be generally wedged shape having a pointed end 352 and an opposed wide end 354. The wedge or augment 330 may have spaced apart upper and lower surfaces 356 and 358 respectively.

The surfaces 356 and 358 may define an included angle αα there between. The angle αα may be selected to provide the proper support underneath the articulating member 308 to compensate for bone loss of the talus 5. The angle αα may for example, be from 5 to 20 degrees. The wedge or augment 330 may include a length L and a width W selected for replacement for the bone loss to the talus 5.

It should be appreciated that in addition to the augment 330 additional augments can be provided as part of a kit to accommodate various amounts of bone loss to the talus. For example, the prosthesis 300 may further include a second augment 334 shown in phantom. The second augment 334 may be defined as an included angle ααα which may be greater or less than the angle αα of the augment 330. It should be appreciated that the second augment 334 may be used alone or that the second augment 334 may be used in combination with the first augment 330.

Augments 330 and 334 may have any suitable size and may include an attachment mechanism for attaching the augment 330 and 334 to either articulating component 308 or to the anchoring member 310 or to both. The attachment mechanism for the augment 330 or for 334 may have any suitable configuration and may, for example be in the form of a groove, a slot, or any other type of mechanical link.

For example and is shown in FIG. 15A, the present invention may be in the form of a prosthesis 300A. The prosthesis 300A may include a talar assembly 304A having an articulating member 308A and an anchoring member 310A. An augment 330A may be physically captured featured between the anchoring member 310A and the articulating member 308A. For example and is shown in FIG. 15A, the augment 330A may include a central opening 360A which mates with outer periphery 362A of the anchoring member 310A. The outer periphery 362A of the anchoring member 310A and the opening 360A of the augment 330A may include a mating taper fit to securely position the augment 330A. In addition to the augment 330A the prosthesis 300A of FIG. 15A may include a second augment 334A having a different size and shape but being fitable to the articulating member 308A and the anchoring member 310A.

Yet another embodiment of the present invention is shown in FIG. 15B as prosthesis 300B. Prosthesis 300B includes yet another form of an augment in the form of augment 330B. The augment 330B includes an opening 360B in which a fastener in form of a screw 364B slideable fits. The fastener 364B may be fitted to threaded opening 362B in the articulating member 308B. While prosthesis 300B may further include a second augment 334B which would be threadably secured with the fastener 364B or a larger fastener 366B to the articulating member 308B of the prosthesis 300B. The augments 334B may have different sizes and shapes to accommodate difference amounts of bone loss to the talus.

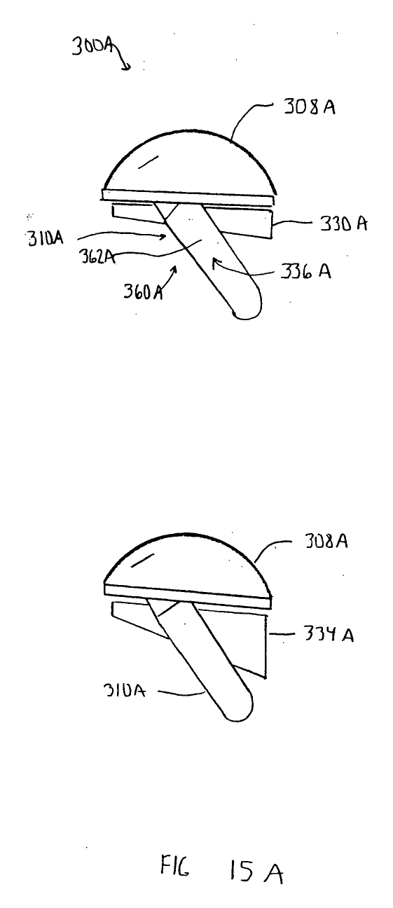

Referring now to FIG. 16, yet another embodiment of the present invention is shown as prosthesis 400. The prosthesis 400 includes a talar assembly 404 similar to the talar assembly 104 of FIGS. 8-13, but includes a connecting means between the anchoring member 410 and the articulating member 408 which is different than that of the prosthesis 100 of FIGS. 8-13.

For example and is shown in FIG. 16 the anchoring member 410 includes a protrusion 412, which is generally cylindrical. The protrusion 412 mattingly fits into a cylindrical cavity 414 formed in the articulating member 408. The anchoring member 410 may be shrunk or press fitted into the cavity 414 of the articulating member 408 to provide for a secure fit.

Referring now to FIG. 17, another embodiment of the present invention is shown as prosthesis 500. The prosthesis 500 includes a talar assembly 504 which is generally similar to the talar assembly 104 of the prosthesis 100 of the prosthesis of FIGS. 8-13 except that that talar assembly 504 includes a different connection mechanism. The talar assembly 504 includes an anchoring member 510 having a protrusion 512 including external threads 515. The talar assembly 504 further includes an articulating member 508 having an internal cavity 514 within which internal threads 513 are formed. The internal threads 513 mate with the external threads 515 of the anchoring member 510 to secure the anchoring member 510 to the articulating member 508.

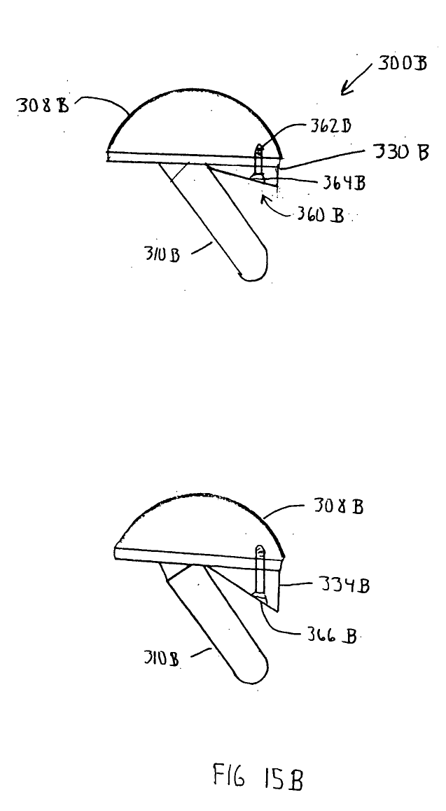

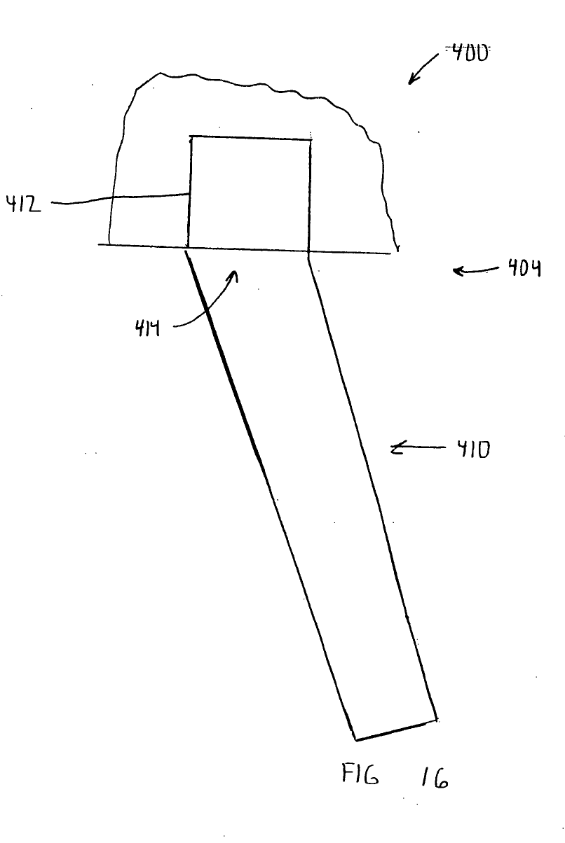

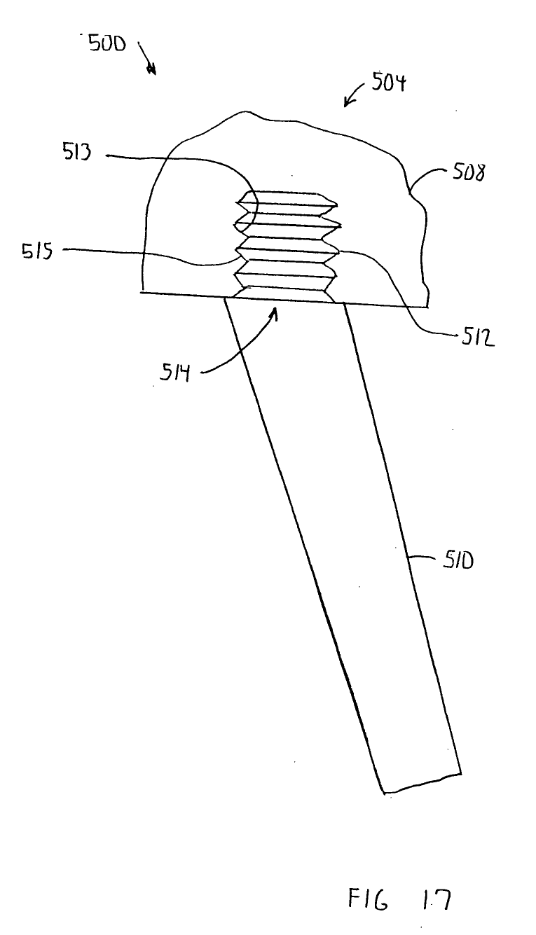

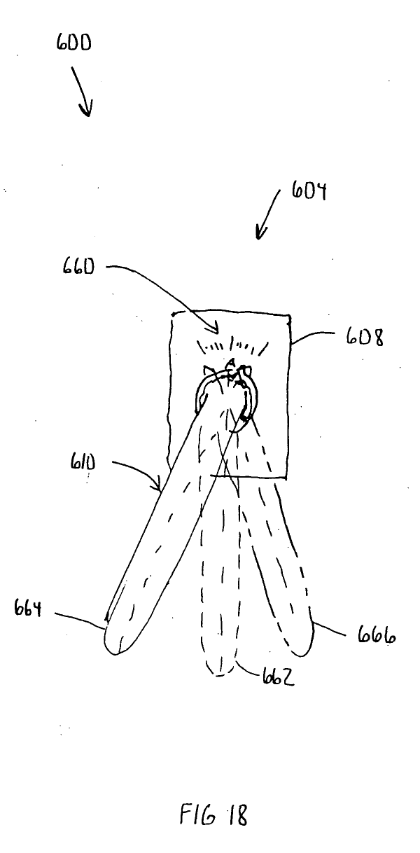

Referring now to FIG. 18, another embodiment of the present invention is shown as implant or prosthesis 600. The prosthesis 600 is similar to the prosthesis 100 of FIG. 8-13 except that the talar assembly 604 of the prosthesis 600 includes an additional feature to assist in the alignment of, for example, indicia 660. The indicia 660 may be positioned on articulating member 608 or on the anchoring member 610, which mates with the articulating member 608 to form the talar assembly 604.

The indicia 660 are used to assist in the aligning of the anchoring member 610 with respect to the articulating member 608. The indicia assists in the proper positioning of the anchoring member 610 to position the anchoring member 610 centrally within the calcaneus 8. Due to the symmetric nature of human body, the anchoring member 610 may be preferably positioned not centrally as shown in dash lines in second position 662, but in one of the first position 664 as shown in solid or in third position 666 as shown in phantom. The first position 664 and the third position 666 correspond to the proper positions of the anchoring member 610 when the prosthesis 600 is positioned on the right ankle or on the left ankle of the patient, respectively.

The indicia 660 may take any form capable of assisting in the alignment of the anchoring member 610 with regard to articulating member 608.

Referring now to FIG. 19, the indicia 660 of the prosthesis 600 is shown in greater detail. The indicia 660 may include a first feature 670 formed on anchoring member 610. The first feature 670 may be in the form of a protrusion or indicator. The indicator 670 may have a distal point or tip 672 for alignment with, for example, second feature 673 associated with the articulating member 608. The second feature 673 may be in the form of a series of radially extending score marks. The score marks 673 may include coarse graduation marks 674 as well as fine graduation marks 676 positioned between the coarse graduation marks 674. Characters 678 may be located on the articulating member 608 and may be associated with the coarse graduation make 674 or the fine graduation make 676 for easier references. The characters 678 may be in the form of for example, numerals or letters.

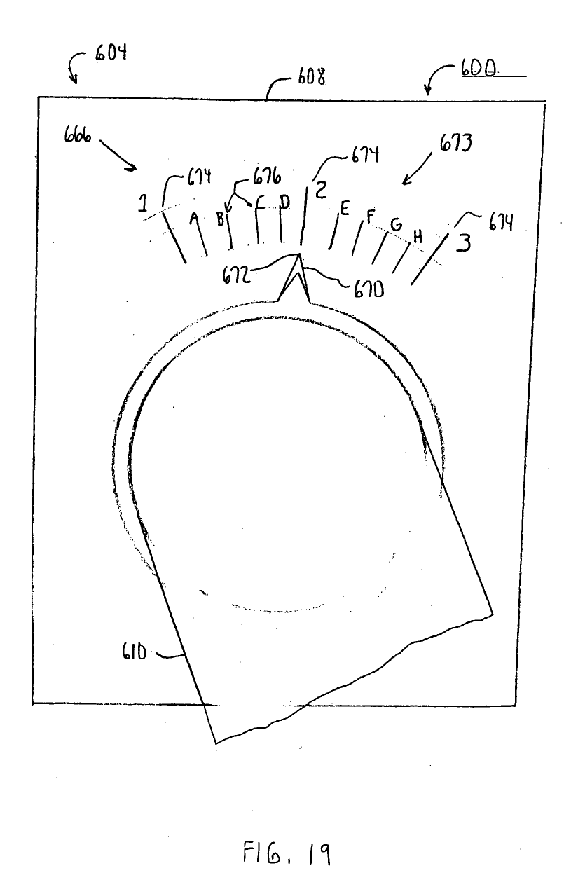

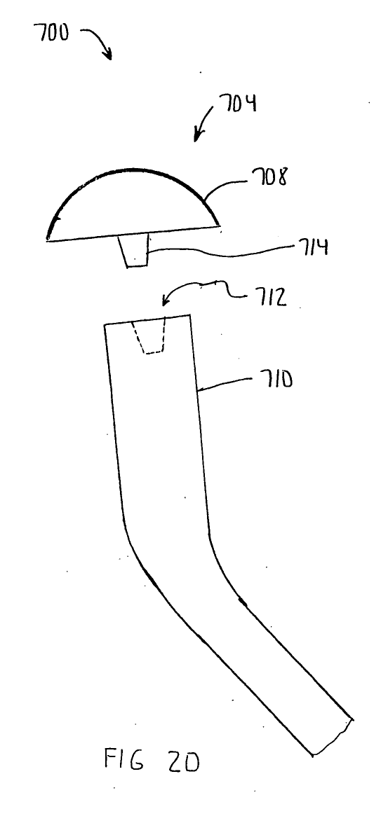

Referring now to FIG. 20, another embodiment of the present invention is shown as implant or prosthesis 700. The prosthesis 700 is similar to the prosthesis 100 of FIGS. 8-13 except that it includes a talar assembly 704 in which the connecting members between the articulating member 704 and the anchoring member 710 are reversed from that of the prosthesis 100 of FIGS. 8-13. For example, and is shown in FIG. 20 the articulating member 708 includes a protrusion 714 which mates with a cavity 712 formed in the anchoring member 710.

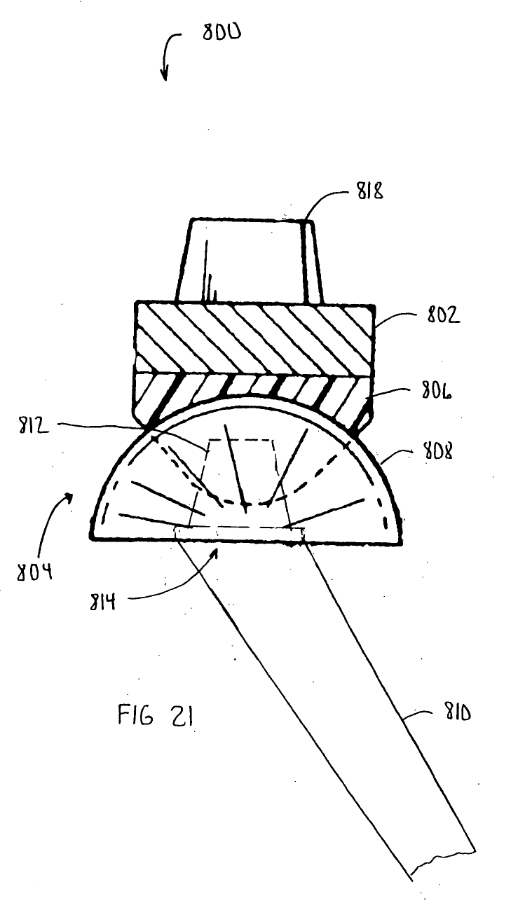

To assist the surgeon in performing in the total ankle arthoplasty revision prosthesis of the present invention, a trial prosthesis 800 is shown in FIG. 21 to be used in conjunction with for example, with the prosthesis 100 of FIGS. 8-13. The trial prosthesis 800 includes components of the same size and shape and more easily removable than those of the implant or prosthesis 100 of FIGS. 8-13. It should be appreciated that the trial prosthesis 800 may include connecting features of somewhat shorter distances or somewhat smaller diameter to provide for an easily removable fit between the trial prosthesis 800 and the cavities or features formed on the patients bone for receiving the trial prosthesis 800.

The trial prosthesis 800 includes a tibia or first member 802 as well as talar assembly 804. The bearing 806 may be placed between the tibia member 802 and the talar assembly 804. It should be appreciated that the bearing 806 can be incorporated into the tibia member 802 or the talar assembly 804 for simplicity. Talar assembly 804 may include an anchoring member 810 and an articulating member 808 to provide for variations in bone loss and to accommodate right and left implants. The anchoring member 810 may include a protrusion 812 which mates with cavity 814 which is formed in the articulating member 808.

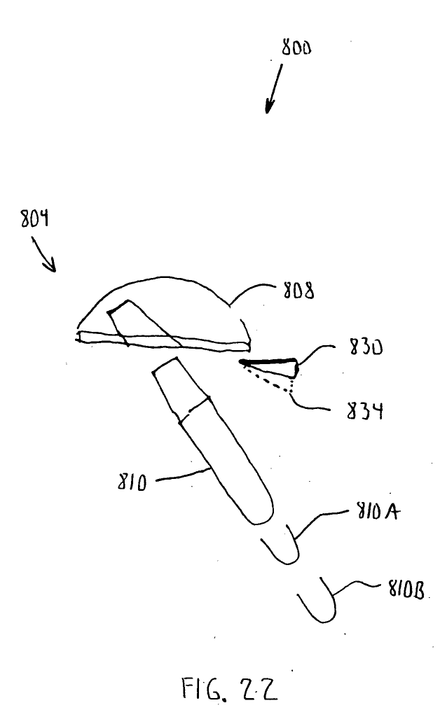

Referring now to FIG. 22, the trial prosthesis 800 may include in addition to the talar assembly 804 having the anchoring 810 and the articulating member 808, a void filling feature 830 in the form of, for example, an augment or a wedge. The augment 830 may have an suitable shape and size.

As is shown in FIG. 22, wedge 830 has a shape similar to the augment or wedge 330 of the implant 300 of FIG. 13. It should be appreciated that a plurality of augments may include for example, an augment 830 and a second augment 834.

It should further be appreciated that the trial 800 may include additional articulating members and anchoring members in addition to the articulating member 808 and the anchoring member 810 is shown in FIG. 22. For example and is shown in FIG. 22, trial 800 may further include a second anchoring member 810A with a size and shape similar to a second anchoring 110A of the prosthesis 100 as shown in FIG. 13. Similarly the trial 800 may include a third anchoring member 810B with a size and shape similar to that of third anchoring member 110B of the prosthesis 100 as shown in FIG. 13.

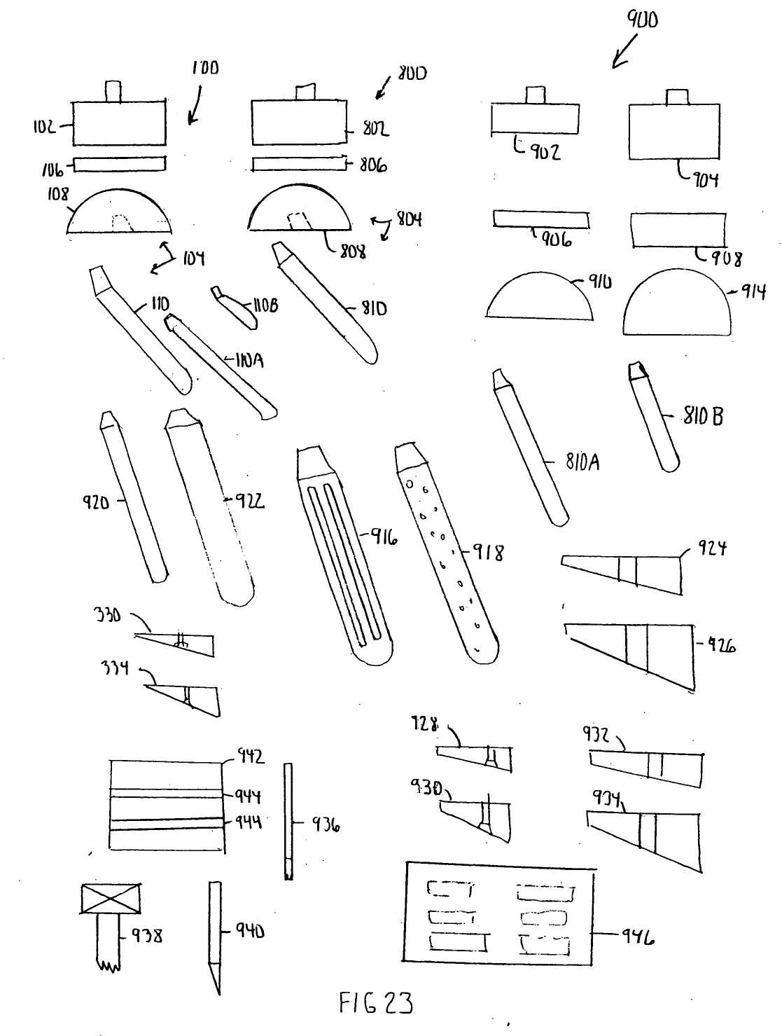

According to the present invention and referring now to FIG. 23, another embodiment of the present invention is shown as prosthetic kit 900. The kit 900 may include an ankle prosthesis 100 including the tibia component 102, the bearing component 106, and the talar assembly 104 including the articulating member 108 and the anchoring member 110. It should be appreciated that the kit 900 may also include a trial 800, for example, the trial tibia component 802, the trial bearing 806, and the trial talar assembly 804 including the trial articulating member 808 and the trial anchoring member 810.

It should further be appreciated that the kit 900 may include additional tibia components for example, second tibia component 902 and third tibia component 904. The second tibia component 902 and third tibia component 904 may have different sizes and shapes but are preferably configured for cooperation with the bearing 106. The kit 900 may further include additional bearings in addition to bearing 106. For example, the kit 900 may include a second bearing 906 as well as a third bearing 908. The additional bearings 906 and 908 are preferably compatiable with tibia component 102 and the articulating member 108.

The kit 900 may further include a plurality of talar assemblies in addition to talar assembly 104. The additional talar assembly may be comprised with additional articulating members or additional anchoring members. For example and is shown in FIG. 23, the kit 900 may include a second articulating member 910 as well as a third articulating member 914.

Similarly, the kit 900 may have additional anchoring members to the anchoring member 110. For example, the kit 900 may include a second anchoring member 110A and a third anchoring member 110B. Further the kit 900 may include yet additional anchoring members. For example, the kit 900 may include a fluted anchoring member 916 or a coated anchoring member 918. Also, the kit 900 may include a thin anchoring member 920 and a thick anchoring member 922. It should be appreciated that for each of the implant components included in the kit 900, a trial component of similar size and shape, should be included in the kit 900 to provide for a full compliment of trials to perform the surgical arthoplasty.

The kit 900 may further include the first augment 330 as well as a second augment 334. It should be appreciated that the kit 900 may further additional augments for example, a third augment 924 and a fourth augment 926. Each of the augments 330, 334, 924, and 926 may have different sizes and shapes. The kit 900 may further include augment trials in the form of for example, first augment trial 928, second augment trial 930, third augment trial 932, and fourth augment trial 934. Kit 900 may further include a plurality of instruments. The instruments may include for example, a mill 936 for example, an end mill for assisting in the preparation of the bone for receiving the implant of the present invention. The kit 900 may further include a solid 938 and an osteotome 940. The instruments such as the mill 936, the saw 938, and the osteotome may be guided or restrained into their proper position by, for example, jig or fixture 942 including features 944 for restraining the instruments.

The instruments and the trials and implants of the kit 900 may be fitted into, for example, a device in the form of a tray 946. The tray 946 may store the instruments, implants, and trials in an organized fashion and provide a carrying device for storing and autoclaving or sterilizing of the instruments and trials. It should be appreciated that the implants may not be included on the tray, but instead be individually packaged and sent to the surgeon based upon optimum selection of the trial located on the tray 946.

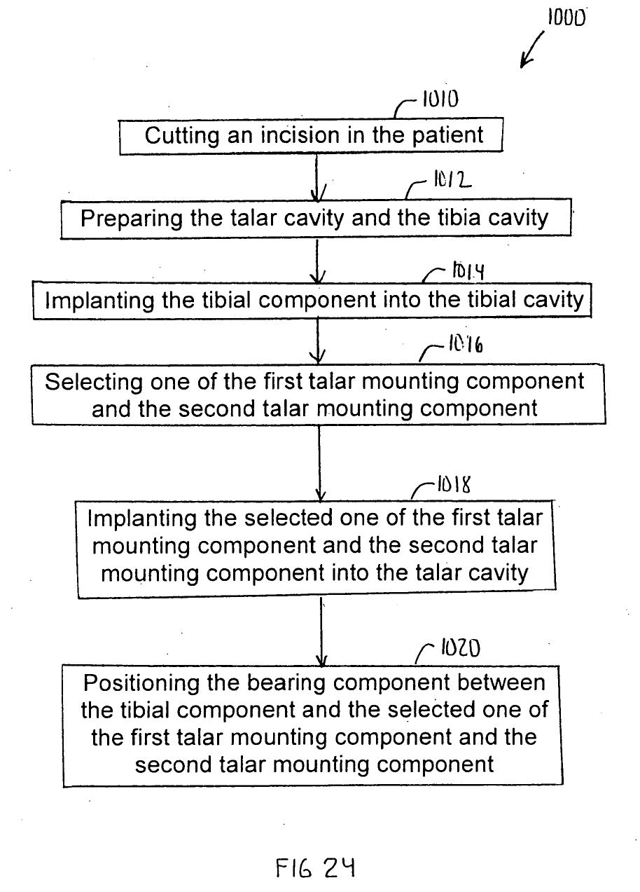

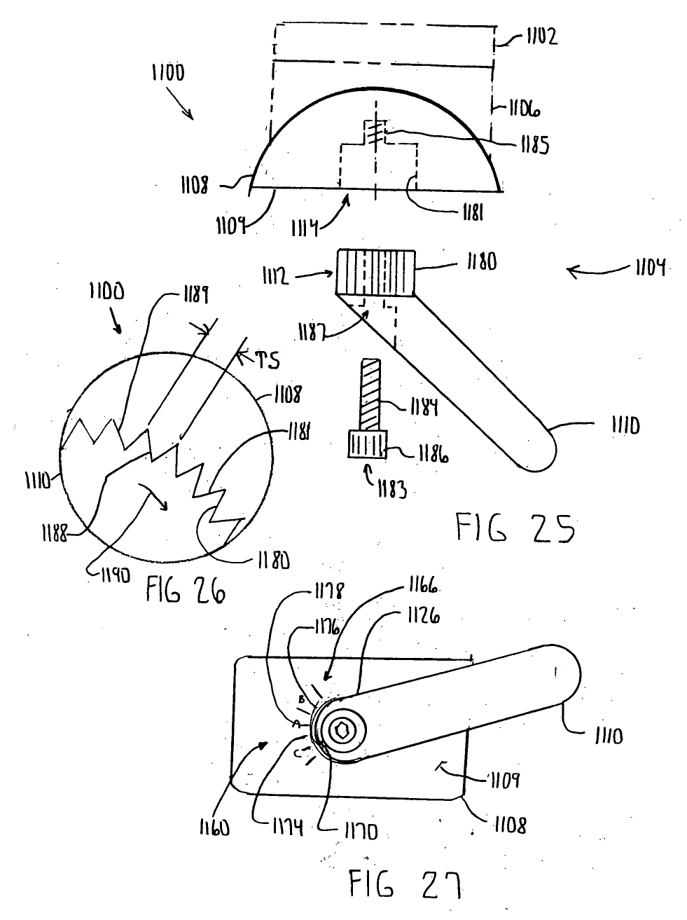

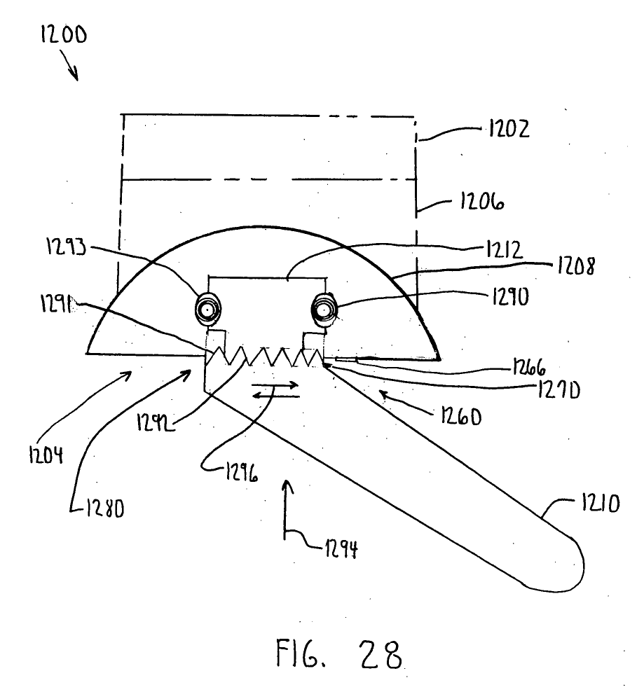

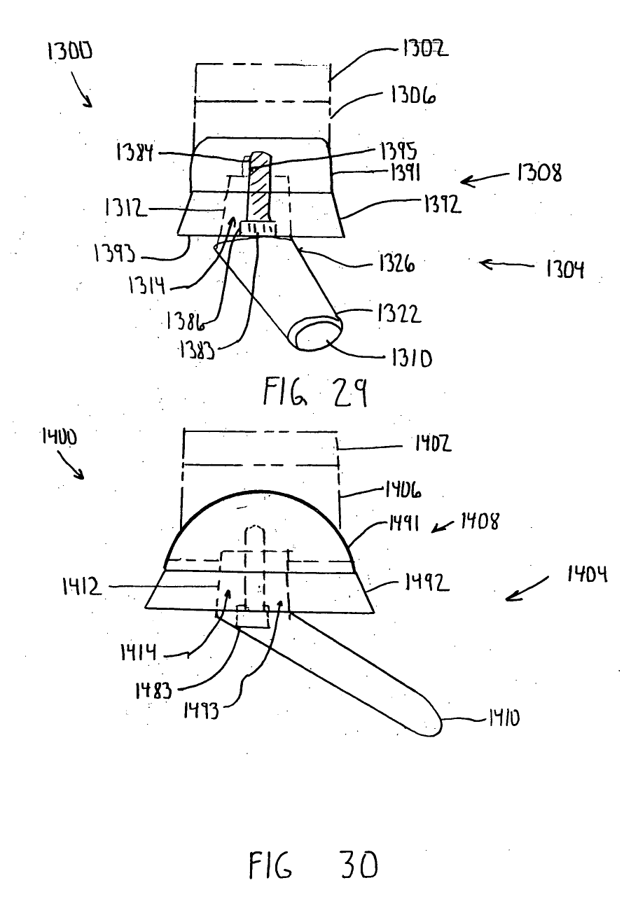

Referring now to FIG. 24, another embodiment of the present invention is shown as method 1000 for providing total ankle arthoplasty. The method 1000 includes a first step 1002 in providing an ankle prosthesis kit including a tibia component, a bearing component, talar articulating component, a first talar mounting component, and a second talar mounting component have at least on dimension different than the first talar component. The method 1000 further includes a second step 1004 of cutting an incision into the patient and a third step 1006 of preparing the talar cavity and the tibia cavity.

The method 1000 may further include a fourth step 1008 of implanting the tibia component into the tibia cavity and a fifth step 1010 of selecting one of the first talar mounting component and the talar component and the second talar mounting component.

The method 1000 may further include a sixth step 1012 of implanting the selected one of the first talar mounting component and the second talar mounting component into the talar cavity. The method may yet further include a seventh step 1014 of positioning the bearing component between the tibia component and selecting one of the first talar mounting component and the second talar mounting component.

Furthering now to FIG. 25, another embodiment of the present invention is shown as ankle prosthesis 1100. Ankle prosthesis 1100 is similar to ankle prosthesis 100 of FIGS. 9-13 except that ankle prosthesis 1100 includes an alternate method of assembling the components of the prosthesis 1100.

As shown in FIG. 25, the ankle prosthesis 1100 includes a tibial member 1102 similar to the tibial member 102 of the prosthesis 100 of FIGS. 9-13 as well as a bearing 1106 similar to the bearing 106 of the ankle prosthesis 100 of FIGS. 9-13. The ankle prosthesis 1100 further includes an articulating member 1108 having an external shape similar to the articulating member 108 of FIGS. 9-13.

The internal configuration of the articulating member of 1108 is somewhat different then the articulating member 108 of the ankle prosthesis 100. The articulating member 1108 includes an internal cavity 1114 to which protrusion 1112 of the anchoring member 1110 matingly fits. The articulating member 1108 and the anchoring member 1110 form the talar assembly 1104. The talar assembly 1104 permits for discrete finite adjustment of the anchoring member 1110 with respect to the articulating member 1108.

As shown in FIG. 25, the protrusion 1112 and the articulating member 1108 adjacent to cavity 1114 include features to provide for the discrete indexing of the anchoring 110 with respect to the articulating member 1108. Such discrete indexing can be accomplished by means of features on the protrusion in the form of, for example, flats, teeth, protrusions or the like. For example, and is shown in FIG. 25, the protrusion 1112 may include external splines. 1118 which mate with internal splines 1181 formed the adjacent the cavity 1114 of the articulating member 1108.

While the protrusion 1112 and the cavity 1118 may provide for the tapered fit and may be a self-locking taper, it should be appreciated that while having features such as the splines 1180 and 1181 located on the protrusion 1112 and the cavity 1118 respectively such a self-locking taper may be difficult to obtain.

For example and is shown in FIG. 25, the internal splines 1181 and the external splines 1180 may have a straight or a non-tapered configuration. A fastener 1183 may be utilized to secure the anchoring member 1110 to the articulating member 1108. For example, the fastener 1183 may be in the form of a socket headed cap screw having a head 1186 which mates with counterbore 1187 formed on the anchoring member 1110. External threads 1184 formed on the fastener 1183 may mate with internal threads 1185 formed on articulating member 1108.

Referring now to FIG. 26, the external spline 1181 formed on the anchoring member 1110 and the internal spline 1180 formed on the articulating member 1108 are shown in greater detail. The external spline 1181 includes a series of external teeth 1189 while the internal spline 1180 includes a series of internal teeth 1188. The number of internal teeth 1188 and the number of external teeth 1189 are preferably the same and the teeth 1188 and 1189 are preferably similar and match or mate with each other.

When the anchoring member 1110 is rotated in the direction of arrow 1190, the anchoring member 1110 may be indexed a distance TS representing the tooth spacing between adjacent teeth 1188 or 1189. The tooth spacing TS may represent an angular indexing of the anchoring member 1110 equal to 360 degrees divided by the number of teeth on the external spline 1180. For example, if there are 72 teeth on the external spline 1180, each index or tooth spacing represents a 5 degree rotation of the anchoring member 1110 with respect to the articulating member 1108.

The applicants have found that the variation from patient to patient of the calcaneous size and position may require the anchoring member to be in a different position with respect to the articulating member. Such variations occur from patient to patient due to the natural anatomy of the patient and due to the progression osteoarthritis and rheumatoid arthritis or due to problems associated with the prior prosthesis.

Further anatomical variations occur between right and left ankles. Therefore, the anchoring member 1110 must be able to be positioned properly with respect to the articulating member 1108. A difference of plus or minus 40 degrees in relative angular orientation and an indexing increment of around 5 degrees may be sufficient to optimize the position of the anchoring member 1110 with respect to the articulating member 1108.

Referring now to FIG. 27, indicia 1160 associated with the ankle prosthesis 1100 are shown in greater detail. The indicia 1160 may include a reference mark 1170 positioned on the proximal portion 1126 of the anchoring member 1110.

The articulating member 1108 may include articulating member indicia 1166 positioned on the bottom surface 1109 of the articulating member 1108. The indicia 1166 may include coarse marks 1174 as well as fine marks 1176 positioned between the coarse marks 1174. Characters 1178 may be associated with the coarse marks 1174 or the fine marks 1176.

Referring now to FIG. 28, a trial 1200 for use with ankle prosthesis 1100 is shown. The trial 1200 includes a tibial trial member 1202 as well as a trial bearing 1206. It should be appreciated that the trial tibia member 1202 and the trial bearing 1206 may be integral with each other. The trial 1200 includes a talar assembly 1204 including an articulating member 1208 as well as an anchoring member 1210.

The trial 1200 includes an adjustable connection 1280 for permitting easy, quick, and accurate positioning of the anchoring member 1210 with respect to the articulating member 1208. The adjustable connection 1280 of the trial 1200 is similar to a connection commonly used in trials that are associated with DePuy S ROM® hip prosthesis trials. Such trials are more fully described in U.S. patent application Ser. No. 10/606,303 filed Jun. 25, 2003, entitled “INSTRUMENT AND ASSOCIATED METHOD OF TRIALING FOR MODULAR HIP STEMS,” hereby incorporated in its entirety by reference.

The adjustable connection 1208 as shown in FIG. 28, includes a helical or angular spring 1290 which may be positioned in a pocket 1293. The pocket 1293 has a generally toroid shape and may for example be in the form of a torus. The helical spring 1290 is used to urge stem 1212 of the anchoring member 1210 in the direction of arrow 1294 in engagement with cavity 1214 of the articulating member 1208.

The helical spring 1290 provides for secure engagement of teeth 1291 located on the anchoring member 1210 with teeth 1292 formed on the articulating member 1208. The helical spring 1290 also permits the separation of the teeth 1291 from the teeth 1292 permitting the anchoring member 1210 to rotate in the direction of arrows 1296 with respect to the articulating member 1208. Indicia 1260 may be utilized to measure the relative location of the anchoring member 1210 with respect to the articulating member 1208. The indicia 1260 may include a mark 1270 located on the anchoring member 1210 and indicia 1266 located on the articulating member 1208.

Yet another embodiment of the present invention is shown as ankle prosthesis 1300 as shown in FIG. 29. The ankle prosthesis 1300 is similar to the ankle prosthesis 100 of FIGS. 9-13 and includes a talar member 1302 similar to the talar 102 of the ankle prosthesis 1300 as well as a bearing 1306 similar to the bearing 106 of the ankle prosthesis 100 for FIGS. 9-13.

The ankle prosthesis 1300 includes an articulating member 1308 somewhat different from the articulating member 108 of the prosthesis 100 of FIGS. 9-13. For example, the articulating member 1308 includes an articulating portion 1391 as well as a support skirt portion 1392. The support skirt portion 1392 may be movable secured to the articulating portion 1391 of the articulating member 1308 of the talar assembly 1304.

The support skirt 1392 provide for an enlarged support face 1393 for contact with the talus. The enlarged support face 1393 assist in minimizing in the subsidence of the ankle prosthesis 1300 into the calcaneuos.

As shown in FIG. 29, the ankle prosthesis 1300 further includes an anchoring member 1310 which is adjustably secured to the articulating member 1308. The anchoring member 1310 includes a distal or calcaneous portion 1322 for engaging with the calcaneous as well as proximal portion 1326. A protrusion 1312 extends proximally from the proximal portion 1326 of the anchoring member 1310. The protrusion 1312 matingly fits with cavity 1314 formed in the support skirt 1392 and in the articulating portion 1391 of the articulating member 1302.

Since the protrusion 1312 as shown in FIG. 29 is tapered, the cavity 1314 of FIG. 29 is also tapered. This tapered connection may be self-locking. The ankle prosthesis 1300, as shown in FIG. 29, may include a fastener 1383 to assist in the securement of the protrusion 1312 to the cavity 1314. The fastener 1383 may be for example, a socket head cap screw and include external threads 1384 which mate with internal threads 1395 formed in the articulating portion 1391 of the articulating member 1308. The fastener 1383 may include a head 1386 which mates with anchoring member 1310 of the talar assembly 1304.

Referring now to FIG. 30, yet another embodiment of the present invention is shown as ankle prosthesis 1400. Ankle prosthesis 1400 is similar to ankle prosthesis 1300 of FIG. 29 and includes a talar assembly 1402 similar to the talar assembly 1302 of FIG. 29. The ankle prosthesis 1400 also includes a bearing 1406 which is similar to the bearing 1306 of the ankle prosthesis 1300 of FIG. 29. The ankle prosthesis 1400 further includes a talar assembly 1404.

The talar assembly 1404 is similar to the talar assembly 1304 of the ankle prosthesis 1300 of FIG. 29 except that the talar assembly 1404 includes a protrusion 1412 which, unlike protrusion 1312 of ankle prosthesis 1300 of FIG. 29, is generally cylindrical. The cavity 1414 which mates with the prosthesis 1412 is also generally cylindrical.

The talar assembly 1404 includes an anchoring member 1410 which is movably positionally attached to the articulating member 1408. A skirt 1492 is positioned between the articulating member 1408 and the anchoring member 1410. Since the protrusion 1412 is generally cylindrical, preferably, a fastener 1483 is used to secure the anchoring member 1410 to the articulating member 1408. The skirt 1492 includes a central opening 1493 through which the protrusion 1412 of the anchoring member 1410 may pass.

Referring now to FIG. 31, yet another embodiment of the present invention is shown as ankle prosthesis 1500. Ankle prosthesis 1500 is similar to the ankle prosthesis 1100 of FIGS. 25-27 and includes a tibial member 1502 as well as a bearing member 1506. The tibial member 1502 and the bearing member 1506 are similar to the talar member 1102 and the bearing member 1106 of the ankle prosthesis 1100 of FIG. 25.

The ankle prosthesis 1500 further includes a talar assembly 1504. The talar assembly 1504 includes an anchoring member 1510 similar to the anchoring member 1110 of prosthesis 1100 of FIG. 25. The talar assembly 1504 further includes an articulating member 1508 which unlike the articulating member 1108 of the ankle prosthesis 1100 of FIG. 25, is modular or has two portions.

For example and is shown in FIG. 31, the articulating member 1208 includes an articulating portion 1596 as well as a support portion 1592. The articulating portion 1596 may be connected, as shown in FIG. 31, to the support portion 1592 by, for example, a dovetail connection 1594. The dovetail connection 1594 may include, as shown in FIG. 31, end portions 1596 formed on the articulating portion 1591 as well as a center portion 1595 located on the support portion 1592.

The ankle prosthesis 1500 of FIG. 31 further includes the anchoring member 1510 which is similar to the anchoring member 1110 of the ankle prosthesis 1100 of FIGS. 25-27. The anchoring member 1510 and the articulating member 1508 are connected by a spline connection 1596. The spline connection 1596 as shown in FIG. 31, includes an external spline 1597 formed on protrusion 1512 of the anchoring member 1510 which mates with an internal spline 1598 formed on cavity 1514 of the articulating portion 1591 of the articulating member 1508. The support portion 1592 may include an opening 1593 to permit the anchoring member 1510 to cooperate with the articulating portion 1591 of articulating member 1508. As shown in FIG. 31, a fastener 1583 may be utilized to secure the anchoring member 1510 to the articulating member 1508.

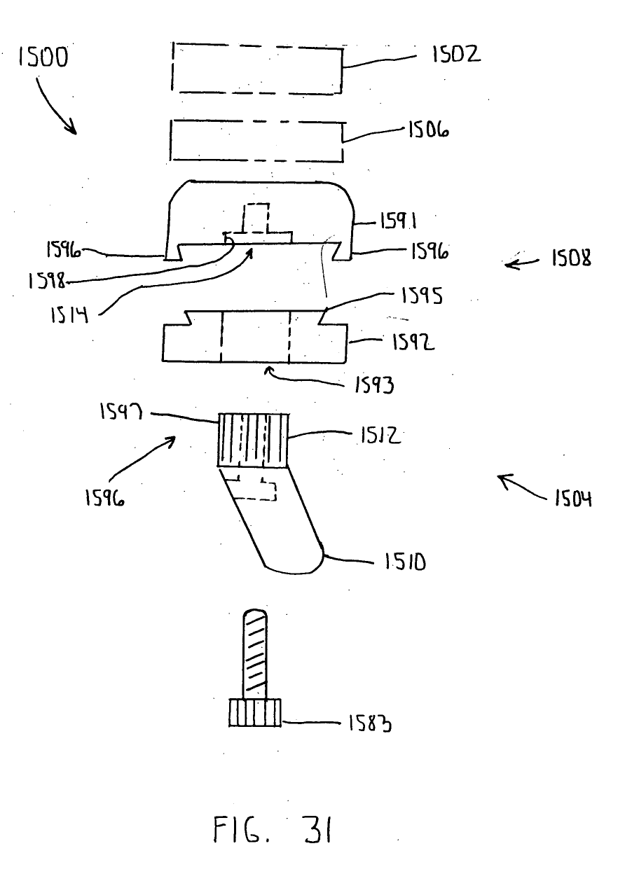

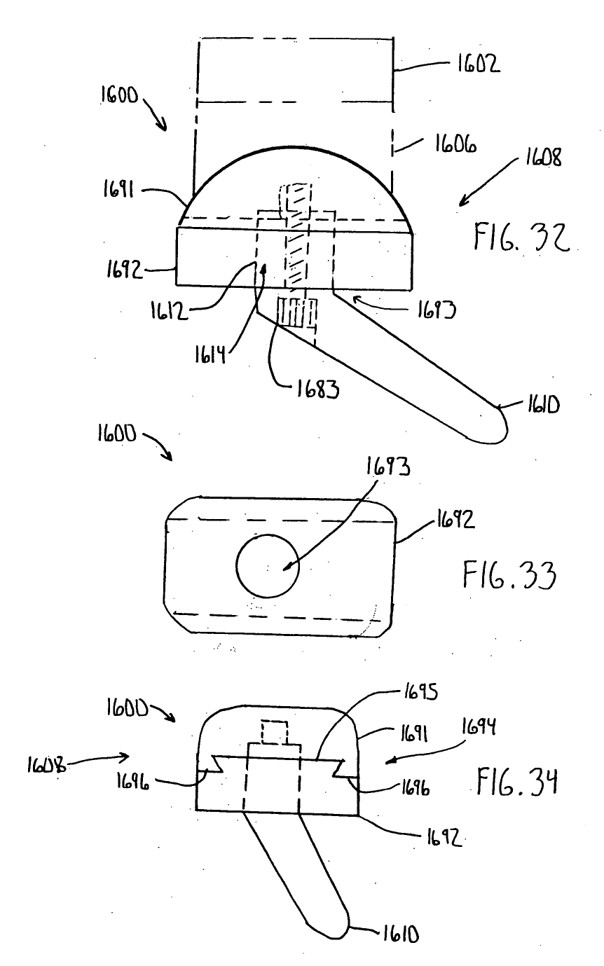

Referring now to FIG. 32-34 yet another embodiment of the present invention is shown as ankle prosthesis 1600. The ankle prosthesis 1600 of FIG. 34 is similar to the ankle prosthesis of 1500 of FIG. 31 except the ankle prosthesis 1600 does not include spline connection. The ankle prosthesis 1600 includes a tibial member 1602 as well as a bearing member 1604. The ankle prosthesis 1600 further includes a talar assembly 1604. The talar assembly 1604 includes an articulating member 1608 as well as an anchoring member 1610.

The articulating member 1608 includes an articulating portion 1691 as well as a support portion 1692. The anchoring member 1610 is connected to the articulating member 1608 by a protrusion 1612 located on the anchoring member 1610 which mates with a cavity 1614 formed in the articulating portion 1691 of the articulating member 1608.

The protrusion 1612 as shown in FIG. 32, is generally cylindrical. An opening 1695 is formed in the support portion 1692 to permit the anchoring member 1610 to pass through the support portion 1692 to secure anchoring member 1610 to the articulating portion 1691 of the articulating member 1608. A fastener 1683 may be used to connect the anchoring member 1610 to the articulating portion 1691 of the articulating member 1608.

Referring now to FIGS. 33 and 34, the support portion 1692 of the articulating member 1608 is secured to the articulating portion 1691 of the articulating member 1608 by means of for example, a dove-tail connection 1694. The dove-tail connection 1694 may include a central protrusion 1695 formed on the support portion 1692 which mates with end protrusion 1696 formed on the articulating portion 1691 of the articulating member 1608.

Although the present invention and its advantages have been described in detail, it should be understood that various changes, substitutions, and alterations can be made therein without departing from the spirit and scope of the present invention as defined by the appended claims.

1. An implant for use in ankle arthroplasty comprising:
a first member for cooperation with the tibia;
a second member for cooperation with the talus and operably associated with the first member; and
a third member rigidly removably connectable to said second member, said third member including a portion thereof for attachment to the calcaneous, said third member adapted to provide for a first position in the calcaneous when said third member is in a first relative position with respect to said second member and to provide for a second position in the calcaneous when said third member is in a second relative position with respect to said second member.

2. The implant of claim 1, wherein one of said second member and said third member comprises a protrusion and wherein the other of said second member and said third member defines a cavity for receiving the protrusion.

3. The implant of claim 2, wherein the protrusion has a periphery a portion of which is conofrustrical.

4. The implant of claim 1, further comprising an augment operably associated with said second member.

5. The implant of claim 4, wherein said augment is fixedly secured to said second member.

6. The implant of claim 4, wherein said augment comprises one of a wedge and a block.

7. The implant of claim 1, further comprising a bearing member positioned between said first member and said second member, said bearing member moveably associated with said first member and with said second member.

8. The implant of claim 1, wherein said second member comprises a first portion operably associated with said first member and a second portion removably attached to said first portion, said second portion for cooperation with the talus.

9. The implant of claim 8, wherein said second portion has an enlarged surface area to provide additional support for the implant on the talus.

10. The implant of claim 8, wherein said second portion is connected to said first portion by a dovetail connection.

11. The implant of claim 1, wherein said third member is connected to said second member by a finitely indexable connection.

12. The implant of claim 11, wherein said finitely indexable connection comprises a spline connection.

13. The implant of claim 1, further comprising a fastener to connect said third member to said second member.

14. The implant of claim 1, further comprising indicia located on at least one of said second member and said third member for assisting in angularly orienting said third member with respect to said second member.

15. A kit for use in assembling an implant for use in ankle arthroplasty comprising:
a first member for cooperation with the tibia;
a second member operably associated with the first member;
a third member rigidly removably connectable to said second member, said third member including a portion thereof for attachment to the talus; and
a fourth member rigidly removably connectable to said second member, said fourth member including a portion thereof for attachment to the talus, the fourth member having at least one dimension different that a dimension of said third member.

16. The kit of claim 10, wherein one of said second member and said third member comprises a protrusion and wherein the other of said second member and said third member defines a cavity for receiving the protrusion.

17. The kit of claim 16, wherein the protrusion has a periphery a portion of which is conofrustrical.

18. The kit of claim 15, further comprising an augment operably associated with said second member.

19. The kit of claim 18, wherein said augment is fixedly secured to said second member.

20. The kit of claim 18, wherein said augment comprises one of a wedge and a block.

21. The kit of claim 15, further comprising a bearing member positioned between said first member and said second member, said bearing member moveably associated with said first member and with said second member.

22. The kit of claim 15, further comprising a tibia connecting member operably associated with the first member.

23. The kit of claim 15, wherein said fourth member includes a portion thereof for attachment to the calcaneus.

24. The kit of claim 15, further including a first trial for performing a trial reduction, the trial adapted to substitute for one of said first member, said second member, said third member and said fourth member.

25. The kit of claim 15, wherein said second member comprises a first portion operably associated with said first member and a second portion removably attached to said first portion, said second portion for cooperation with the talus.

26. The kit of claim 25, wherein said second portion has an enlarged surface area to provide additional support for the implant on the talus.

27. The kit of claim 25, wherein said second portion is connected to said first portion by a dovetail connection.

28. The kit of claim 15, wherein said third member is connected to said second member by a finitely indexable connection.

29. The kit of claim 28, wherein said finitely indexable connection comprises a spline connection.

30. The kit of claim 15, further comprising a fastener to connect said third member to said second member.

31. The kit of claim 15, further comprising indicia located on at least one of said second member and said third member for assisting in angularly orienting said third member with respect to said second member.

32. A talar component for use in an implant for use in ankle arthroplasty comprising:
a first member for cooperation with the talus; and
a second member rigidly removably connectable to said first member, said second member including a portion thereof for attachment to the calcaneous, said second member adapted to provide for a first position in the calcaneous when said second member is in a first relative position with respect to said first member and to provide for a second position in the calcaneous when said second member is in a second relative position with respect to said first member.

33. The talar component of claim 32, wherein one of said first member and said second member comprises a protrusion and wherein the other of said first member and said second member defines a cavity for receiving the protrusion.

34. The talar component of claim 33, wherein said protrusion has a periphery a portion of which is conofrustrical.

35. The talar component of claim 32, further comprising an augment operably associated with said first member.

36. The talar component of claim 35, wherein said augment is fixedly secured to said second member.

37. The talar component of claim 35, wherein said augment comprises one of a wedge and a block.

38. The talar component of claim 32, wherein said first member comprises a first portion and a second portion removably attached to said first portion, said second portion for cooperation with the talus.

39. The talar component of claim 38, wherein said second portion has an enlarged surface area to provide additional support for the implant on the talus.

40. The talar component of claim 38, wherein said second portion is connected to said first portion by a dovetail connection.

41. The talar component of claim 32, wherein said second member is connected to said first member by a finitely indexable connection.

42. The talar component of claim 41, wherein said finitely indexable connection comprises a spline connection.

43. The talar component of claim 32, further comprising a fastener to connect said second member to said first member.

44. The talar component of claim 32, further comprising indicia located on at least one of said first member and said second member for assisting in angularly orienting said second member with respect to said first member.

45. A method for providing ankle arthroplasty comprising:
providing an ankle prosthesis kit including an tibial component, a bearing component, a talar articulating component, a first talar mounting component, and a second talar mounting component having at least one dimension different than the first talar mounting component;
cutting an incision in the patient;
Preparing the talar cavity and the tibia cavity;
implanting the tibial component into the tibial cavity;
selecting one of the first talar mounting component and the second talar mounting component;
implanting the selected one of the first talar mounting component and the second talar mounting component into the talar cavity; and
positioning the bearing component between the tibial component and the selected one of the first talar mounting component and the second talar mounting component.

46. A trial for use with an implant having a first component, a second component and a third component for use in ankle arthroplasty comprising:
a first trial member for cooperation with the tibia, said first trial member corresponding to the first component;
a second trial member operably associated with the first trial member, said second trial member corresponding to the second component; and
a third trial member rigidly removably connectable to said second member, said third trial member including a portion thereof for attachment to the talus, said third trial member corresponding to the third component.
